(19) United States
(12) Patent Application Publication
(10) Pub. No.: US 2024/0374601 A1
(43) Pub. Date: Nov. 14, 2024

(54) METHODS AND COMPOSITIONS FOR TREATING CANCER

(71) Applicant: Onconova Therapeutics, Inc., Newtown, PA (US)

(72) Inventors: Steven M. Fruchtman, New York, NY (US); Matthew Parris, Plainsboro, NJ (US); Mark S. Gelder, Montgomery, TX (US)

(21) Appl. No.: 18/664,568

(22) Filed: May 15, 2024

Related U.S. Application Data
(63) Continuation of application No. PCT/US22/50503, filed on Nov. 18, 2022.
(60) Provisional application No. 63/280,957, filed on Nov. 18, 2021.

(51) Int. Cl. A61K 31/519; A61K 9/00; A61K 45/06; A61P 35/00

(52) U.S. Cl. CPC A61K 31/519; A61K 9/0053; A61K 45/06; A61P 35/00

(57) ABSTRACT

Disclosed herein is a method of treating ovarian cancer by administering to the subject a solid pharmaceutical composition comprising 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof, alone or in combination with a second agent such as letrozole.

### CROSS-REFERENCE

This application is a continuation of International Application PCT/US22/50503 filed Nov. 18, 2022, which claims the benefit of U.S. Provisional Application No. 63/280,957, filed Nov. 18, 2021, each of which is entirely incorporated by reference herein.

### BACKGROUND

Ovarian cancer ranks fifth in cancer deaths among women. A large subset of ovarian cancers have alterations in both the phosphatidylinositol-4,5-bisphosphate 3-kinase (PI3K) and mitogen-activated protein kinase (MAPK) pathways.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY OF THE INVENTION

In some embodiments, the present disclosure provides a method of treating low grade serous ovarian cancer in a subject in need thereof, the method comprising administering to the subject a therapeutically-effective amount of a compound of formula (I)

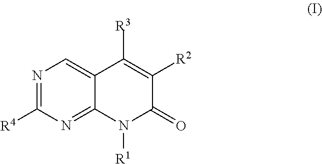

wherein:
   R¹ is alkyl, alkenyl, alkynyl, cycloalkyl, aryl, heteroaryl sor heterocyclyl, each of which is unsubstituted or substituted, or hydrogen;
   R² is alkyl, alkenyl, alkynyl, cycloalkyl, aryl, heteroaryl, heterocyclyl, —CN, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen;
   R³ is alkyl, alkenyl, alkynyl, cycloalkyl, aryl, heterocyclyl or heteroaryl, each of which is unsubstituted or substituted, or hydrogen or halogen;
   R⁴ is alkyl, alkenyl, alkynyl, cycloalkyl, aryl, heterocyclyl, heteroaryl, —CN, C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
   each R⁵ and R⁶ is independently alkyl, alkenyl, alkynyl, cycloalkyl, aryl, heterocyclyl, or heteroaryl, each of which is independently substituted or unsubstituted, or hydrogen or halogen:
provided that, when R⁴ is —NR⁵R⁶ and one of R⁵ and R⁶ is pyridyl, then R² is not —C(O)CH₃,
or a pharmaceutically-acceptable salt thereof.

In some embodiments, the present disclosure provides a method of treating high grade serous ovarian cancer in a subject in need thereof, the method comprising administering to the subject a therapeutically-effective amount of a compound of formula (I)

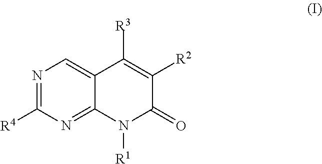

wherein:
   R¹ is alkyl, alkenyl, alkynyl, cycloalkyl, aryl, heteroaryl or heterocyclyl, each of which is unsubstituted or substituted, or hydrogen;
   R² is alkyl, alkenyl, alkynyl, cycloalkyl, aryl, heteroaryl, heterocyclyl, —CN, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen;
   R³ is alkyl, alkenyl, alkynyl, cycloalkyl, aryl, heterocyclyl or heteroaryl, each of which is unsubstituted or substituted, or hydrogen or halogen;
   R⁴ is alkyl, alkenyl, alkynyl, cycloalkyl, aryl, heterocyclyl, heteroaryl, —CN, C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
   each R⁵ and R⁶ is independently alkyl, alkenyl, alkynyl, cycloalkyl, aryl, heterocyclyl, or heteroaryl, each of which is independently substituted or unsubstituted, or hydrogen or halogen;
provided that, when R⁴ is —NR⁵R⁶ and one of R⁵ and R⁶ is pyridyl, then R² is not —C(O)CH₃,
or a pharmaceutically-acceptable salt thereof.

In some embodiments, the present disclosure provides a method of treating ovarian cancer, e.g., serous ovarian cancer, e.g., low grade serous ovarian cancer in a subject in need thereof, the method comprising administering to the subject a therapeutically-effective amount of a compound of formula (I)

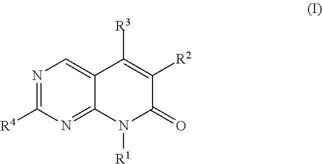

wherein:
   R¹ is alkyl, alkenyl, alkynyl, cycloalkyl, aryl, heteroaryl or heterocyclyl, each of which is unsubstituted or substituted, or hydrogen;
   R² is alkyl, alkenyl, alkynyl, cycloalkyl, aryl, heteroaryl, heterocyclyl, —CN, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen;
   R³ is alkyl, alkenyl, alkynyl, cycloalkyl, aryl, heterocyclyl or heteroaryl, each of which is unsubstituted or substituted, or hydrogen or halogen;
   R⁴ is alkyl, alkenyl, alkynyl, cycloalkyl, aryl, heterocyclyl, heteroaryl, —CN, C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
   each R⁵ and R⁶ is independently alkyl, alkenyl, alkynyl, cycloalkyl, aryl, heterocyclyl, or heteroaryl, each of which is independently substituted or unsubstituted, or hydrogen or halogen;
provided that, when R⁴ is —NR⁵R⁶ and one of R⁵ and R⁶ is pyridyl, then R² is not —C(O)CH₃,
or a pharmaceutically-acceptable salt thereof;
wherein the subject received a therapy other than the compound for the ovarian cancer, e.g., serous ovarian cancer, e.g., low grade serous ovarian cancer prior to the administering.

In some embodiments, the present disclosure provides a method of treating ovarian cancer; e.g., serous ovarian cancer; e.g., low grade serous ovarian cancer in a subject in need thereof, the method comprising administering to the subject a therapeutically-effective amount of a compound of formula (I)

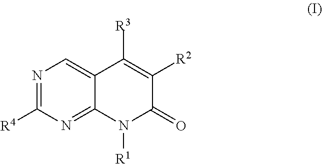

wherein:
   R¹ is alkyl, alkenyl, alkynyl, cycloalkyl, aryl, heteroaryl or heterocyclyl, each of which is unsubstituted or substituted, or hydrogen;
   R² is alkyl, alkenyl, alkynyl, cycloalkyl, aryl, heteroaryl, heterocyclyl, —CN, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen;
   R³ is alkyl, alkenyl, alkynyl, cycloalkyl, aryl, heterocyclyl or heteroaryl, each of which is unsubstituted or substituted, or hydrogen or halogen;
   R⁴ is alkyl, alkenyl, alkynyl, cycloalkyl, aryl, heterocyclyl, heteroaryl, —CN, C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
   each R⁵ and R⁶ is independently alkyl, alkenyl, alkynyl, cycloalkyl, aryl, heterocyclyl, or heteroaryl, each of which is independently substituted or unsubstituted, or hydrogen or halogen;
provided that, when R⁴ is —NR⁵R⁶ and one of R⁵ and R⁶ is pyridyl, then R² is not —C(O)CH₃,
or a pharmaceutically-acceptable salt thereof;
wherein the administering is once daily for at least 4 weeks.

In some embodiments, the present disclosure provides a method of treating ovarian cancer; e.g., serous ovarian cancer; e.g., low grade serous ovarian cancer in a subject in need thereof, the method comprising administering to the subject a therapeutically-effective amount of a compound of formula (I)

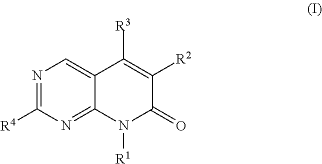

wherein:
   R¹ is alkyl, alkenyl, alkynyl, cycloalkyl, aryl, heteroaryl or heterocyclyl, each of which is unsubstituted or substituted, or hydrogen.
   R² is alkyl, alkenyl, alkynyl, cycloalkyl, aryl, heteroaryl, heterocyclyl, —CN, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen;
   R³ is alkyl, alkenyl, alkynyl, cycloalkyl, aryl, heterocyclyl or heteroaryl, each of which is unsubstituted or substituted, or hydrogen or halogen;
   R⁴ is alkyl, alkenyl, alkynyl, cycloalkyl, aryl, heterocyclyl, heteroaryl, —CN, C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
   each R⁵ and R⁶ is independently alkyl, alkenyl, alkynyl, cycloalkyl, aryl, heterocyclyl, or heteroaryl, each of which is independently substituted or unsubstituted, or hydrogen or halogen;
provided that, when R⁴ is —NR⁵R⁶ and one of R⁵ and R⁶ is pyridyl, then R² is not —C(O)CH₃, or a pharmaceutically-acceptable salt thereof;
wherein the administering is a 4-week cycle of:
   (i) a continuous, three-week period of once-daily administration; and
   ii) immediately following the three-week period, one week of no administration.

In some embodiments, the present disclosure provides a method of treating ovarian cancer; e.g., serous ovarian cancer; e.g., low grade serous ovarian cancer in a subject in need thereof, the method comprising
(i) administering to the subject a therapeutically-effective amount of a first compound,
wherein the first compound is a compound of formula (I):

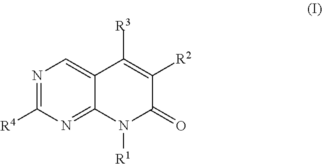

wherein:
   R¹ is alkyl, alkenyl, alkynyl, cycloalkyl, aryl, heteroaryl or heterocyclyl, each of which is unsubstituted or substituted, or hydrogen;
   R² is alkyl, alkenyl, alkynyl, cycloalkyl, aryl, heteroaryl, heterocyclyl, —CN, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵ or —OS(O)₂R⁵, each of which is independently substituted or unsubstituted, or hydrogen or halogen;
   R³ is alkyl, alkenyl, alkynyl, cycloalkyl, aryl, heterocyclyl or heteroaryl, each of which is unsubstituted or substituted, or hydrogen or halogen;
   R⁴ is alkyl, alkenyl, alkynyl, cycloalkyl, aryl, heterocyclyl, heteroaryl, —CN, C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
   each R⁵ and R⁶ is independently alkyl, alkenyl, alkynyl, cycloalkyl, aryl, heterocyclyl, or heteroaryl, each of which is independently substituted or unsubstituted, or hydrogen or halogen;
provided that, when R⁴ is —NR⁵R⁶ and one of R⁵ and R⁶ is pyridyl, then R² is not —C(O)CH₃,
or a pharmaceutically-acceptable salt thereof; and
   (ii) administering to the subject a therapeutically-effective amount of a second compound.

In some embodiments, the present disclosure provides a method of treating ovarian cancer; e.g., serous ovarian cancer; e.g., low grade serous ovarian cancer in a subject in need thereof, the method comprising orally administering to the subject a solid pharmaceutical composition, the solid pharmaceutical composition comprising 40 mg to 500 mg of a compound that is 8-cyclopentyl-2-((4-(4-methylpiperazin-1-yl)phenyl)amino)-7 oxo-7,8-dihydropyrido[2,3-d]pyrimidine-6-carbonitrile monolactate, wherein the subject received a therapy other than the compound for the ovarian cancer; e.g., serous ovarian cancer; e.g., low grade serous ovarian cancer prior to the administering, wherein the therapy was received after the subject was diagnosed with ovarian cancer; e.g., serous ovarian cancer; e.g., low grade serous ovarian cancer, and wherein the subject has not responded to the therapy prior to the administering; and wherein the administering comprises 3 weeks of once-daily administration.

In some embodiments, the present disclosure provides a method of treating ovarian cancer; e.g., serous ovarian cancer; e.g., low grade serous ovarian cancer in a subject in need thereof, the method comprising (i) orally administering to the subject a solid pharmaceutical composition, wherein the solid pharmaceutical composition comprises 40 mg to 500 mg of a compound that is 8-cyclopentyl-2-((4-(4-methylpiperazin-1-yl)phenyl)amino)-7-oxo-7,8-dihydropyrido[2,3-d]pyrimidine-6-carbonitrile monolactate, wherein the subject received a therapy other than the compound for the ovarian cancer; e.g., serous ovarian cancer; e.g., low grade serous ovarian cancer prior to the administering, wherein the therapy was received after the subject was diagnosed with ovarian cancer; e.g., serous ovarian cancer; e.g., low grade serous ovarian cancer, and wherein the subject has not responded to the therapy prior to the administering; and wherein the administering comprises 3 weeks of once-daily administration; and (ii) orally administering to the subject a therapeutically-effective amount of letrozole.

In some embodiments, the present disclosure provides a method of treating ovarian cancer; e.g., serous ovarian cancer; e.g., low grade serous ovarian cancer in a subject in need thereof, the method comprising (i) orally administering to the subject a solid pharmaceutical composition, wherein the solid pharmaceutical composition comprises 40 mg to 500 mg of a compound that is 8-cyclopentyl-2-((4-(4-methylpiperazin-1-yl)phenyl)amino)-7-oxo-7,8-dihydropyrido[2,3-d]pyrimidine-6-carbonitrile monolactate, wherein the subject received a therapy other than the compound for the ovarian cancer; e.g., serous ovarian cancer; e.g., low grade serous ovarian cancer prior to the administering, wherein the therapy was received after the subject was diagnosed with ovarian cancer; e.g., serous ovarian cancer; e.g., low grade serous ovarian cancer, and wherein the subject has not responded to the therapy prior to the administering, and wherein the administering comprises 3 weeks of once-daily administration; and (ii) orally administering to the subject a therapeutically-effective amount of megestrol acetate.

### BRIEF DESCRIPTION OF THE DRAWINGS

FIG. 1 illustrates kinome tree for CDK 4/6 inhibitors: Compound 1, Comparator 1, Comparator 2, Comparator 3

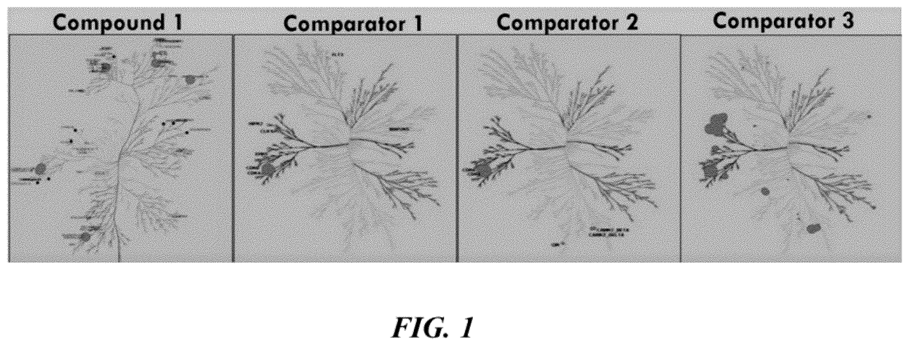

FIG. 2 shows reduced neutropenia in ovarian cancer cell line treated with Compound 1 compared to the ovarian cancer cell line treated with Comparator 1.

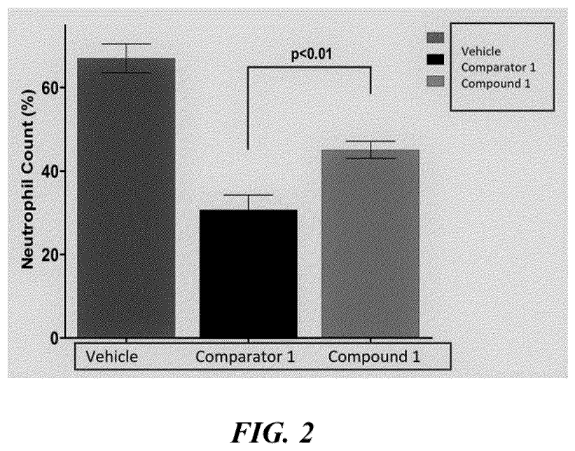

FIG. 3 shows a higher inhibition of ovarian cancer cell line growth with Compound 1 compared to Comparator 1.

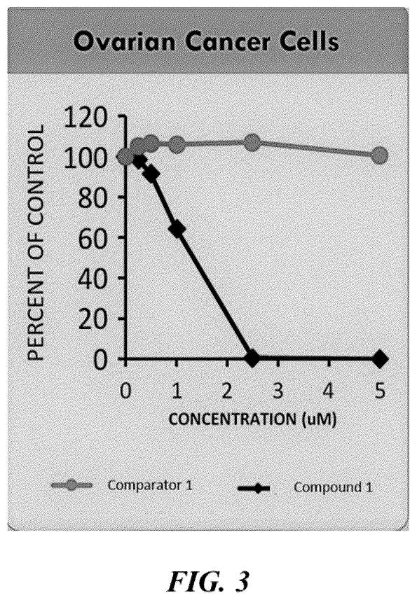

### DETAILED DESCRIPTION OF THE INVENTION

Provided herein are compositions and methods for treating ovarian cancer, e.g., serous ovarian cancer, e.g., low grade serous ovarian cancer by administering to a subject in need thereof a pharmaceutical composition, the pharmaceutical composition comprising in a unit dosage form a therapeutically-effective amount of a compound described herein or a pharmaceutically-acceptable salt thereof. In some embodiments, the methods further comprise administering a second pharmaceutical composition comprising in a unit dosage form a therapeutically-effective amount of a second compound, for example an estrogen blocking drug.

### Compounds of the Disclosure

A compound disclosed herein can be of the formula:

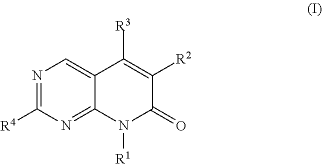

wherein:
   R¹ is alkyl, alkenyl, alkynyl, cycloalkyl, aryl, heteroaryl or heterocyclyl, each of which is unsubstituted or substituted, or hydrogen;
   R² is alkyl, alkenyl, alkynyl, cycloalkyl, aryl, heteroaryl, heterocyclyl, —CN, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen;
   R³ is alkyl, alkenyl, alkynyl, cycloalkyl, aryl, heterocyclyl or heteroaryl, each of which is unsubstituted or substituted, or hydrogen or halogen;
   R⁴ is alkyl, alkenyl, alkynyl, cycloalkyl, aryl, heterocyclyl, heteroaryl, —CN, C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
   each R⁵ and R⁶ is independently alkyl, alkenyl, alkynyl, cycloalkyl, aryl, heterocyclyl, or heteroaryl, each of which is independently substituted or unsubstituted, or hydrogen or halogen;
provided that, when R⁴ is —NR⁵R⁶ and one of R⁵ and R⁶ is pyridyl, then R² is not —C(O)CH₃,
or a pharmaceutically-acceptable salt thereof.

A compound disclosed herein can be of the formula:

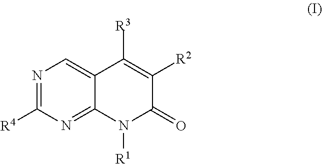

wherein:
   R¹ is alkyl, alkenyl, alkynyl, cycloalkyl, aryl, heteroaryl or heterocyclyl, each of which is unsubstituted or substituted, or hydrogen;
   R² is alkyl, alkenyl, alkynyl, cycloalkyl, aryl, heteroaryl, heterocyclyl, —CN, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen;
   R³ is C₂-C₆ alkyl, alkenyl, alkynyl, cycloalkyl, aryl, heterocyclyl or heteroaryl, each of which is unsubstituted or substituted, or hydrogen or halogen;
   R⁴ is alkyl, alkenyl, alkynyl, cycloalkyl, aryl, heterocyclyl, heteroaryl, —CN, C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
   each R⁵ and R⁶ is independently alkyl, alkenyl, alkynyl, cycloalkyl, aryl, heterocyclyl, or heteroaryl, each of which is independently substituted or unsubstituted, or hydrogen or halogen,
or a pharmaceutically-acceptable salt thereof.

A compound disclosed herein can be a pharmaceutically-acceptable salt of the formula:

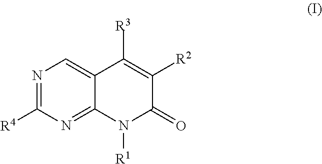

wherein:
   R¹ is alkyl, alkenyl, alkynyl, cycloalkyl, aryl, heteroaryl or heterocyclyl, each of which is unsubstituted or substituted, or hydrogen;
   R² is alkyl, alkenyl, alkynyl, cycloalkyl, aryl, heteroaryl, heterocyclyl, —CN, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen;
   R³ is alkyl, alkenyl, alkynyl, cycloalkyl, aryl, heterocyclyl or heteroaryl, each of which is unsubstituted or substituted, or hydrogen or halogen;
   R⁴ is alkyl, alkenyl, alkynyl, cycloalkyl, aryl, heterocyclyl, heteroaryl, —CN, C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
   each R⁵ and R⁶ is independently alkyl, alkenyl, alkynyl, cycloalkyl, aryl, heterocyclyl, or heteroaryl, each of which is independently substituted or unsubstituted, or hydrogen or halogen.

A compound disclosed herein can be a lactate salt of the formula:

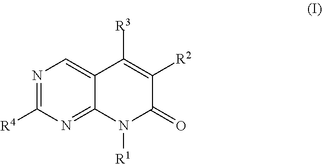

wherein:
   R¹ is alkyl, alkenyl, alkynyl, cycloalkyl, aryl, heteroaryl or heterocyclyl, each of which is unsubstituted or substituted, or hydrogen;
   R² is alkyl, alkenyl, alkynyl, cycloalkyl, aryl, heteroaryl, heterocyclyl, —CN, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen;
   R³ is alkyl, alkenyl, alkynyl, cycloalkyl, aryl, heterocyclyl or heteroaryl, each of which is unsubstituted or substituted, or hydrogen or halogen;
   R⁴ is alkyl, alkenyl, alkynyl, cycloalkyl, aryl, heterocyclyl, heteroaryl, —CN, C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
   each R⁵ and R⁶ is independently alkyl, alkenyl, alkynyl, cycloalkyl, aryl, heterocyclyl, or heteroaryl, each of which is independently substituted or unsubstituted, or hydrogen or halogen.

In some embodiments, R¹ is cycloalkyl. In some embodiments, R¹ is C₃-C₈ cycloalkyl. In some embodiments, R¹ is an unsubstituted cyclopentyl. In some embodiments, R¹ is an unsubstituted cyclopentyl. In some embodiments, R² is CN. In some embodiments, R³ is hydrogen.

In some embodiments, R⁴ is —NR⁵R⁶. In some embodiments, one of R⁵ and R⁶ is hydrogen. In some embodiments, one of R⁵ and R⁶ is phenyl. In some embodiments, one of R⁵ and Re is phenyl substituted with heterocyclyl. In some embodiments, one of R⁵ and R⁶ is phenyl substituted with heterocyclyl, wherein the heterocyclyl contains at least one ring nitrogen atom. In some embodiments, one of R⁵ and Re is phenyl substituted with C₃-C₈ heterocyclyl. In some embodiments, one of R⁵ and R⁶ is phenyl substituted with C₆ heterocyclyl. In some embodiments, one of R⁵ and R⁶ is phenyl substituted with piperazinyl, wherein the piperazinyl is unsubstituted or substituted. In some embodiments, one of R⁵ and R⁶ is phenyl substituted with piperazinyl, wherein the piperazinyl is substituted with an alkyl. In some embodiments, one of R³ and R⁶ is phenyl substituted with 4-methyl piperazinyl.

In some embodiments, R⁴ is

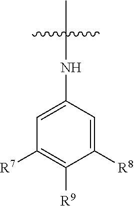

wherein:
R⁷ is alkyl, alkenyl, alkynyl, cycloalkyl, aryl, heterocyclyl, heteroaryl, —OR⁵, —SR⁵, or —NR⁵R⁶, each of which is unsubstituted or substituted, or hydrogen;
R⁸ is alkyl, alkenyl, alkynyl, cycloalkyl, aryl, heterocyclyl, heteroaryl, —OR⁵, —SR⁵, or —NR⁵R⁶, each of which is unsubstituted or substituted, or hydrogen; and
R⁹ is alkyl, alkenyl, alkynyl, cycloalkyl, aryl, heterocyclyl, heteroaryl, —OR⁵, —SR⁵, or —NR⁵R⁶, each of which is unsubstituted or substituted, or hydrogen.

In some embodiments, R⁷ is hydrogen. In some embodiments, R⁸ is hydrogen. In some embodiments, R⁹ unsubstituted or substituted heterocyclyl. In some embodiments, R⁹ is unsubstituted or substituted piperazinyl. In some embodiments, R⁹ is piperazinyl substituted with alkyl. In some embodiments, R⁹ is 4-methyl piperazinyl.

In some embodiments, the compound is a compound of formula (II)

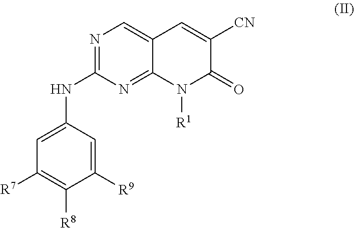

In some embodiments, the compound is a compound of formula (III)

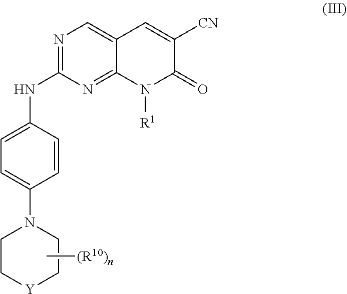

wherein:
Y is O, S, or NR¹¹;
each R¹⁰ is independently alkyl, alkenyl, alkynyl, cycloalkyl, aryl, heterocyclyl, heteroaryl, —OR⁵, —SR⁵, or —NR⁵R⁶, each of which is unsubstituted or substituted;
R¹¹ is alkyl, alkenyl, alkynyl, cycloalkyl, aryl, heterocyclyl, or heteroaryl, each of which is independently substituted or unsubstituted, or hydrogen or halogen; and
n is 0, 1, 2, 3, 4, 5, 6, 7, or 8.

In some embodiments, R¹ is cycloalkyl. In some embodiments, R¹ is C₃-C₈ cycloalkyl. In some embodiments, R¹ is an unsubstituted cyclopentyl. In some embodiments, R¹ is a unsubstituted cyclopentyl.

In some embodiments, Y is NR¹¹. In some embodiments, R¹¹ is alkyl. In some embodiments, R¹¹ is methyl. In some embodiments, n is 0.

In some embodiments, Y is NR¹¹. In some embodiments, R¹¹ is alkyl. In some embodiments, R¹¹ is methyl. In some embodiments, n is 0.

In some embodiments, the compound is of the formula:

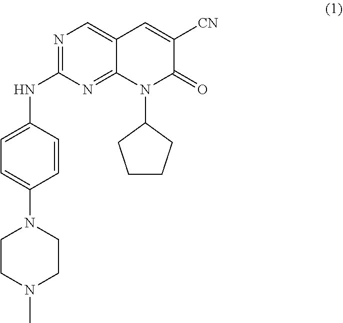 [SMILES: CN1CCN(c2ccc(Nc3ncc4cc(C#N)c(=O)n(C5CCCC5)c4n3)cc2)CC1]

8-cyclopentyl-2-((4-(4-methylpiperazin-1-yl)phenyl)amino)-7-oxo-7,8-dihydropyrido[2,3-d]pyrimidine-6-carbonitrile, or a pharmaceutically-acceptable salt thereof.

In some embodiments, the compound is in the form of a salt formed by combining a compound with lactic acid. In some embodiments, a compound disclosed herein is 8-cyclopentyl-2-((4-(4-methylpiperazin-1-yl)phenyl)amino)-7-oxo-7,8-dihydropyrido[2,3-d]pyrimidine-6-carbonitrile monolactate.

Several moieties described herein may be substituted or unsubstituted. Non-limiting examples of optional substituents include hydroxyl groups, sulfhydryl groups, halogens, amino groups, nitro groups, nitroso groups, cyano groups, azido groups, sulfoxide groups, sulfone groups, sulfonamide groups, carboxyl groups, carboxaldehyde groups, imine groups, alkyl groups, halo-alkyl groups, alkenyl groups, halo-alkenyl groups, alkynyl groups, halo-alkynyl groups, alkoxy groups, aryl groups, aryloxy groups, aralkyl groups, arylalkoxy groups, heterocyclyl groups, acyl groups, acyloxy groups, carbamate groups, amide groups, ureido groups, epoxy groups, and ester groups.

Non-limiting examples of alkyl groups include straight, branched, and cyclic alkyl groups. An alkyl group can be, for example, a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted.

Non-limiting examples of straight alkyl groups include methyl, ethyl, propyl, butyl, pentyl, hexyl, heptyl, octyl, nonyl, and decyl.

Branched alkyl groups include any straight alkyl group substituted with any number of alkyl groups. Non-limiting examples of branched alkyl groups include isopropyl, isobutyl, sec-butyl, and t-butyl.

Non-limiting examples of substituted alkyl groups includes hydroxymethyl, chloromethyl, trifluoromethyl, aminomethyl, 1-chloroethyl, 2-hydroxyethyl, 1,2-difluoroethyl, and 3-carboxypropyl.

Non-limiting examples of cyclic alkyl groups include cyclopropyl, cyclobutyl, cyclopentyl, cyclohexyl, cycloheptlyl, and cyclooctyl groups. Cyclic alkyl groups also include fused-, bridged-, and spiro-bicycles and higher fused-, bridged-, and spiro-systems. A cyclic alkyl group can be substituted with any number of straight, branched, or cyclic alkyl groups. Non-limiting examples of cyclic alkyl groups include cyclopropyl, 2-methyl-cycloprop-1-yl, cycloprop-2-en-1-yl, cyclobutyl, 2,3-dihydroxycyclobut-1-yl, cyclobut-2-en-1-yl, cyclopentyl, cyclopent-2-en-1-yl, cyclopenta-2,4-dien-1-yl, cyclohexyl, cyclohex-2-en-1-yl, cycloheptyl, cyclooctanyl, 2,5-dimethylcyclopent-1-yl, 3,5-dichlorocyclohex-1-yl, 4-hydroxycyclohex-1-yl, 3,3,5-trimethylcyclohex-1-yl, octahydropentalenyl, octahydro-1H-indenyl, 3a,4,5,6,7,7a-hexahydro-3H-inden-4-yl, decahydroazulenyl, bicyclo-[2,1,1]hexanyl, bicyclo[2,2,1]heptanyl, bicyclo[3,1,1]heptanyl, 1,3-dimethyl[2,2,1]heptan-2-yl, bicyclo[2,2,2]octanyl, and bicyclo[3,3,3]undecanyl.

Non-limiting examples of alkenyl and alkenylene groups include straight, branched, and cyclic alkenyl groups. The olefin or olefins of an alkenyl group can be, for example, E, Z, cis, trans, terminal, or exo-methylene. An alkenyl or alkenylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of alkenyl and alkenylene groups include ethenyl, prop-1-en-1-yl, isopropenyl, but-1-en-4-yl: 2-chloroethenyl, 4-hydroxy buten-1-yl, 7-hydroxy-7-methyloct-4-en-2-yl, and 7-hydroxy-7-methyloct-3,5-dien-2-yl.

Non-limiting examples of alkynyl or alkynylene groups include straight, branched, and cyclic alkynyl groups. The triple bond of an alkylnyl or alkynylene group can be internal or terminal. An alkylnyl or alkynylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of alkynyl or alkynylene groups include ethynyl, prop-2-yn-1-yl, prop-1-yn-1-yl, and 2-methyl-hex-4-yn-1-yl: 5-hydroxy-5-methylhex-3-yn-1-yl, 6-hydroxy-6-methylhept-3-yn-2-yl, and 5-hydroxy-5-ethylhept-3-yn-1-yl.

A halo-alkyl group can be any alkyl group substituted with any number of halogen atoms, for example, fluorine, chlorine, bromine, and iodine atoms. A halo-alkenyl group can be any alkenyl group substituted with any number of halogen atoms. A halo-alkynyl group can be any alkynyl group substituted with any number of halogen atoms.

An alkoxy group can be, for example, an oxygen atom substituted with any alkyl, alkenyl, or alkynyl group. An ether or an ether group comprises an alkoxy group. Non-limiting examples of alkoxy groups include methoxy, ethoxy, propoxy, isopropoxy, and isobutoxy.

An aryl group can be heterocyclic or non-heterocyclic. An aryl group can be monocyclic or polycyclic. An aryl group can be substituted with any number of substituents described herein, for example, hydrocarbyl groups, alkyl groups, alkoxy groups, and halogen atoms. Non-limiting examples of aryl groups include phenyl, toluyl, naphthyl, pyrrolyl, pyridyl, imidazolyl, thiophenyl, and furyl. Non-limiting examples of substituted aryl groups include 3,4-dimethylphenyl, 4-tert-butylphenyl, 4-cyclopropylphenyl, 4-diethylaminophenyl, 4-(trifluoromethyl)phenyl, 4-(difluoromethoxy)-phenyl, 4-(trifluoromethoxy)phenyl, 3-chlorophenyl, 4-chlorophenyl, 3,4-dichlorophenyl, 2-fluorophenyl, 2-chlorophenyl, 2-iodophenyl, 3-iodophenyl, 4-iodophenyl, 2-methylphenyl, 3-fluorophenyl, 3-methylphenyl, 3-methoxyphenyl, 4-fluorophenyl, 4-methylphenyl, 4-methoxyphenyl, 2,3-difluorophenyl, 3,4-difluorophenyl, 3,5-difluorophenyl, 2,3-dichlorophenyl, 3,4-dichlorophenyl, 3,5-dichlorophenyl. 2-hydroxyphenyl, 3-hydroxyphenyl, 4-hydroxyphenyl, 2-methoxyphenyl, 3-methoxyphenyl, 4-methoxyphenyl, 2,3-dimethoxyphenyl, 3,4-dimethoxyphenyl, 3,5-dimethoxyphenyl, 2,4-difluorophenyl, 2,5-difluorophenyl, 2,6-difluorophenyl, 2,3,4-trifluorophenyl, 2,3,5-trifluorophenyl, 2,3,6-trifluorophenyl, 2,4,5-trifluorophenyl, 2,4,6-trifluorophenyl, 2,4-dichlorophenyl, 2,5-dichlorophenyl, 2,6-dichlorophenyl, 3,4-dichlorophenyl, 2,3,4-trichlorophenyl, 2,3,5-trichlorophenyl, 2,3,6-trichlorophenyl, 2,4,5-trichlorophenyl, 3,4,5-trichlorophenyl, 2,4,6-trichlorophenyl, 2,3-dimethylphenyl, 2,4-dimethylphenyl, 2,5-dimethylphenyl, 2,6-dimethylphenyl, 2,3,4-trimethylphenyl, 2,3,5-trimethylphenyl, 2,3,6-trimethylphenyl, 2,4,5-trimethylphenyl, 2,4,6-trimethylphenyl, 2-ethylphenyl, 3-ethylphenyl, 4-ethylphenyl, 2,3-diethylphenyl, 2,4-diethylphenyl, 2,5-diethylphenyl, 2,6-diethylphenyl, 3,4-diethylphenyl, 2,3,4-triethylphenyl, 2,3,5-triethylphenyl, 2,3,6-triethylphenyl, 2.4.5-triethylphenyl, 2,4,6-triethylphenyl, 2-isopropylphenyl, 3-isopropylphenyl, and 4-isopropylphenyl.

Non-limiting examples of substituted aryl groups include 2-aminophenyl, 2-(N-methylamino)phenyl, 2-(N,N-dimethylamino)phenyl, 2-(N-ethylamino)phenyl, 2-(N,N-diethylamino)phenyl, 3-aminophenyl, 3-(N-methylamino)phenyl, 3-(N,N-dimethylamino)phenyl, 3-(N-ethylamino)phenyl, 3-(N,N-diethylamino)phenyl, 4-aminophenyl, 4-(N-methylamino)phenyl, 4-(N,N-dimethylamino)phenyl, 4-(N-ethylamino)phenyl, and 4-(N,N-diethylamino)phenyl.

A heterocycle can be any ring containing a ring atom that is not carbon, for example, N, O, S, P, Si, B, or any other heteroatom. A heterocycle can be substituted with any number of substituents, for example, alkyl groups and halogen atoms. A heterocycle can be aromatic (heteroaryl) or non-aromatic. Non-limiting examples of heterocycles include pyrrole, pyrrolidine, pyridine, piperidine, succinamide, maleimide, morpholine, imidazole, thiophene, furan, tetrahydrofuran, pyran, and tetrahydropyran.

Non-limiting examples of heterocycles (heterocyclyl) include: heterocyclic units having a single ring containing one or more heteroatoms, non-limiting examples of which include, diazirinyl, aziridinyl, azetidinyl, pyrazolidinyl, imidazolidinyl, oxazolidinyl, isoxazolinyl, thiazolidinyl, isothiazolinyl, oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, piperidin-2-onyl, 2,3,4,5-tetrahydro-1H-azepinyl, 2,3-dihydro-1H-indole, and 1,2,3,4-tetrahydroquinoline; and ii) heterocyclic units having 2 or more rings one of which is a heterocyclic ring, non-limiting examples of which include hexahydro-1H-pyrrolizinyl. 3a,4,5,6,7,7a-hexahydro-1H-benzo[d]imidazolyl, 3a,4,5,6,7,7a-hexahydro-1H-indolyl, 1,2,3,4-tetrahydroquinolinyl, and decahydro-1H-cycloocta[b]pyrrolyl.

Non-limiting examples of heteroaryl include: i) heteroaryl rings containing a single ring, non-limiting examples of which include, 1,2,3,4-tetrazolyl, [1.2.3]triazolyl, [1,2,4]triazolyl, triazinyl, thiazolyl, 1H-imidazolyl, oxazolyl, isoxazolyl, isothiazolyl, furanyl, thiophenyl, pyrimidinyl, 2-phenylpyrimidinyl, pyridinyl, 3-methylpyridinyl, and 4-dimethylaminopyridinyl; and ii) heteroaryl rings containing 2 or more fused rings one of which is a heteroaryl ring, non-limiting examples of which include: 7H-purinyl, 9H-purinyl, 6-amino-9H-purinyl, 5H-pyrrolo[3,2-d]pyrimidinyl, 7H-pyrrolo[2,3-d]pyrimidinyl, pyrido[2,3-d]pyrimidinyl, 4,5,6,7-tetrahydro-1-H-indolyl, quinoxalinyl, quinazolinyl, quinolinyl, 8-hydroxy-quinolinyl, and isoquinolinyl.

Any compound herein can be purified. A compound herein can be least 1% pure, at least 2% pure, at least 3% pure, at least 4% pure, at least 5% pure, at least 6% pure, at least 7% pure, at least 8% pure, at least 9% pure, at least 10% pure, at least 11% pure, at least 12% pure, at least 13% pure, at least 14% pure, at least 15% pure, at least 16% pure, at least 17% pure, at least 18% pure, at least 19% pure, at least 20% pure, at least 21% pure, at least 22% pure, at least 23% pure, at least 24% pure, at least 25% pure, at least 26% pure, at least 27% pure, at least 28% pure, at least 29% pure, at least 30% pure, at least 31% pure, at least 32% pure, at least 33% pure, at least 34% pure, at least 35% pure, at least 36% pure, at least 37% pure, at least 38% pure, at least 39% pure, at least 40% pure, at least 41% pure, at least 42% pure, at least 43% pure, at least 44% pure, at least 45% pure, at least 46% pure, at least 47% pure, at least 48% pure, at least 49% pure, at least 50% pure, at least 51% pure, at least 52% pure, at least 53% pure, at least 54% pure, at least 55% pure, at least 56% pure, at least 57% pure, at least 58% pure, at least 59% pure, at least 60% pure, at least 61% pure, at least 62% pure, at least 63% pure, at least 64% pure, at least 65% pure, at least 66% pure, at least 67% pure, at least 68% pure, at least 69% pure, at least 70% pure, at least 71% pure, at least 72% pure, at least 73% pure, at least 74% pure, at least 75% pure, at least 76% pure, at least 77% pure, at least 78% pure, at least 79% pure, at least 80% pure, at least 81% pure, at least 82% pure, at least 83% pure, at least 84% pure, at least 85% pure, at least 86% pure, at least 87% pure, at least 88% pure, at least 89% pure, at least 90% pure, at least 91% pure, at least 92% pure, at least 93% pure, at least 94% pure, at least 95% pure, at least 96% pure, at least 97% pure, at least 98% pure, at least 99% pure, at least 99.1% pure, at least 99.2% pure, at least 99.3% pure, at least 99.4% pure, at least 99.5% pure, at least 99.6% pure, at least 99.7% pure, at least 99.8% pure, or at least 99.9% pure.

### Pharmaceutically Acceptable Salts

The method disclosed herein provides the use of pharmaceutically-acceptable salts of any compound described herein. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base.

Acid addition salts can arise from the addition of an acid to a compound disclosed herein. In some embodiments, the acid is organic. In some embodiments, the acid is inorganic. In some embodiments, the acid is lactic acid, salicylic acid, tartaric acid, ascorbic acid, gentisinic acid, gluconic acid, glucaronic acid, saccaric acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid citric acid, oxalic acid, maleic acid, hydrochloric acid, hydrobromic acid, hydroiodic acid, nitric acid, nitrous acid, sulfuric acid, sulfurous acid, a phosphoric acid, or isonicotinic acid. In some embodiments, the salt is an acid addition salt with lactic acid. In some embodiments, the salt is an acid addition salt of 8-cyclopentyl-2-((4-(4-methylpiperazin-1-yl)phenyl)amino)-7-oxo-7,8-dihydropyrido[2,3-d]pyrimidine-6-carbonitrile with lactic acid.

In some embodiments, the salt is a lactate salt, a salicylate salt, a tartrate salt, an ascorbate salt, a gentisinate salt, a gluconate salt, a glucaronate salt, a saccarate salt, a formate salt, a benzoate salt, a glutamate salt, a pantothenate salt, an acetate salt, a propionate salt, a butyrate salt, a fumarate salt, a succinate salt, a methanesulfonate salt, an ethanesulfonate salt, a benzenesulfonate salt, a p-toluenesulfonate salt, a citrate salt, an oxalate salt, a maleate salt, hydrochloride salt, a hydrobromide salt, a hydroiodide salt, a nitrate salt, a nitrite salt, a sulfate salt, a sulfite salt, a phosphate salt, isonicotinate salt. In some embodiments, the salt is a lactate salt. In some embodiments, the salt is a monolactate salt. In some embodiments, the compound is 8-cyclopentyl-2-((4-(4-methylpiperazin-1-yl)phenyl)amino)-7-oxo-7,8-dihydropyrido[2,3-d]pyrimidine-6-carbonitrile monolactate.

Metal salts can arise from the addition of an inorganic base to a compound disclosed herein. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

### Therapeutic Methods

In one aspect, the disclosure provides a method to treat a disease by administering a compound disclosed herein, e.g., compound 1. In some embodiments, the disease is a cancer, e.g., ovarian cancer, e.g., serous ovarian cancer, e.g., low grade serous ovarian cancer. In some embodiments, the ovarian cancer, e.g., serous ovarian cancer, e.g., low grade serous ovarian cancer is associated with dysregulation of one or more of Membrane receptor tyrosine kinases (RTK). Phosphoinositide 3-kinase (PI3 kinase or PI3K), protein kinase B (AKT), and mitogen-activated protein kinase (MAPK) pathways. In some embodiments, the ovarian cancer, e.g., serous ovarian cancer, e.g., low grade serous ovarian cancer is associated with one or more of KRAS, BRAF and NRAS mutation. In some embodiments, the ovarian cancer, e.g., serous ovarian cancer, e.g., low grade serous ovarian cancer is associated with an exon 2 KRAS mutation. In some embodiments, the ovarian cancer, e.g., serous ovarian cancer, e.g., low grade serous ovarian cancer is associated with one or more KRAS mutation e.g., KRASG12V, KRASG12D, KRASG12R/C/A/S, KRASG13C, KRASQ61K,H,L. In some embodiments, the ovarian cancer, e.g., serous ovarian cancer, e.g., low grade serous ovarian cancer is associated with an BRAF mutation, e.g., codon 600 of exon 15, BRAFV600E. In some embodiments, the ovarian cancer, e.g., serous ovarian cancer, e.g., low grade serous ovarian cancer is associated with an NRAS mutation, e.g., NRASQ61K, NRASQ61R. In some embodiments, the ovarian cancer, e.g., serous ovarian cancer, e.g., low grade serous ovarian cancer is associated with low frequency gene alterations, e.g., USP9X, EIFIAX, MAP2K1, RAS GAP, NF1, ERRB2 and BRAF fusions, MACF1, ARIDIA, NF2, DOTIL and ASHIL gene alteration. In some embodiments, the ovarian cancer, e.g., serous ovarian cancer, e.g., low grade serous ovarian cancer is associated with gene copy number aberrations. In some embodiments, the ovarian cancer, e.g., serous ovarian cancer, e.g., low grade serous ovarian cancer is associated with TP53 mutations. In some embodiments, the ovarian cancer, e.g., serous ovarian cancer, e.g., low grade serous ovarian cancer is not associated with TP53 mutations. In some embodiments, the ovarian cancer, e.g., serous ovarian cancer, e.g., low grade serous ovarian cancer is associated with one or more of BRCA1, BRCA2, BRIP1, CHEK2, RAD51C mutations. In some embodiments, the ovarian cancer, e.g., serous ovarian cancer, e.g., low grade serous ovarian cancer is not associated with BRCA1, BRCA2, BRIP1, CHEK2, RAD51C mutations.

In some embodiments, the ovarian cancer, e.g., serous ovarian cancer, e.g., low grade serous ovarian cancer is a hormone receptor positive (HR+) cancer. In some embodiments, the hormone receptor is an estrogen receptor. In some embodiments, the hormone receptor is a progesterone receptor. In some embodiments, the ovarian cancer, e.g., serous ovarian cancer, e.g., low grade serous ovarian cancer is an estrogen receptor positive (ER+) low grade serous ovarian cancer. In some embodiments, the ovarian cancer, e.g., serous ovarian cancer, e.g., low grade serous ovarian cancer is a progesterone receptor positive (PR+) low grade serous ovarian cancer. In some embodiments, the ovarian cancer, e.g., serous ovarian cancer, e.g., low grade serous ovarian cancer is an estrogen receptor positive, progesterone receptor positive (ER+/PR+) low grade serous ovarian cancer.

In some embodiments, a compound described herein, e.g., compound 1 can modulate, e.g., downmodulate these kinase pathways, or a portion thereof, for example, cyclin-dependent kinases (CDK). In some embodiments, overexpression of CDK, e.g., CDK 4/6 causes cell-cycle deregulation in cancers, e.g., ovarian cancer, e.g., serous ovarian cancer, e.g., low grade serous ovarian cancer. In some embodiments, modulation of kinase pathways can result in the obstruction of proliferation signal receipt in cells, thus arresting tumor growth.

In some embodiments, a compound described herein can be an inhibitor of the cyclin-dependent kinase protein 4 (CDK4). In some embodiments, a compound described herein can be an inhibitor of cyclin-dependent kinase protein 6 (CDK6). In some embodiments, a compound described herein can be an inhibitor of CDK1. In some embodiments, a compound described herein can be an inhibitor of CDK2. In some embodiments, a compound described herein can be an inhibitor of CDK7. In some embodiments, a compound described herein can be an inhibitor of CDK9. In some embodiments, a compound described herein can be an inhibitor of CDK16. In some embodiments, a compound described herein can be an inhibitor of CDK17. In some embodiments, a compound described herein can be an inhibitor of CDK 4/6.

In some embodiments, a compound described herein can be used for treating a cancer e.g., ovarian cancer, e.g., serous ovarian cancer, e.g., low grade serous ovarian cancer where other CDK4/6 inhibitors have failed. In some embodiments, a compound described herein can be an inhibitor of colony stimulating factor receptor 1 (CSFIR). In some embodiments, a compound described herein can be an inhibitor of proto-oncogene receptor tyrosine kinase KIT. In some embodiments, a compound described herein is an inhibitor of the AMPK-related protein kinase 5 (ARK5 or NUAK1) protein. ARK5 regulates Atk-dependent cell survival and migration (e.g., formation of metastases) through inhibition of cellular metabolism. ARK5 overexpression is found in multiple tumors and is associated with poor prognosis in metastatic breast cancer, multiple myeloma, and hepatocellular carcinoma. In some embodiments, inhibition of ARK5 induces cell death through PI3K/AKT/mTOR pathway. In some embodiments, a combination of CDK and ARK5 inhibitors has a synergistic effect on cancer cells by simultaneously inhibiting cell cycle (cytostatic) and cellular metabolism (cytotoxic) through CDK and ARK5, respectively.

In some embodiments, the inclusion of ARK5 in the functional activity profile of a compound described herein overcomes the emergence of resistance to CDK4/6 inhibitors due to the loss of retinoblastoma function and C-Myc overexpression. C-Myc expression is dependent on ARK5 activity and is involved in the pathogenesis of certain tumors. In some embodiments, the dual inhibitory effect of a compound described herein provides a therapeutic strategy to optimize efficacy of CDK 4/6 inhibition and reduce emergence of resistance. The present disclosure provides a method for the use of a compound disclosed herein, for example, for treating cancer, e.g., ovarian cancer, e.g., serous ovarian cancer, e.g., low grade serous ovarian cancer.

In some embodiments, a compound disclosed herein, e.g., 8-cyclopentyl-2-((4-(4-methylpiperazin-1-yl)phenyl)amino)-7-oxo-7,8-dihydropyrido[2,3-d]pyrimidine-6-carbonitrile (Compound 1) or a pharmaceutically-acceptable salt thereof, may be active in numerous tumor types with acceptable and differentiated safety profile. In preclinical models. Compound 1 caused less myelosuppression and less neutropenia in comparison to an FDA approved CDK 4/6 inhibitor (Comparator-1 in TABLE 1). Compound 1 inhibited growth of cancer cell lines, e.g., breast cancer cells, ovarian cancer cells, and prostate cancer cells, which are resistant to Comparator-1. Compound 1 also had comparable IC₅₀ values for CDK4 and CDK6 as FDA approved CDK 4/6 inhibitor (Comparator-1. Comparator-2, and Comparator-3 in TABLE 1). Compound 1 had significantly lower IC₅₀ values (1 to 3 magnitude lower) for CSFIR, ARK 5, KIT, than Comparator-1. Comparator-2, and Comparator-3. Compound 1 had significantly lower IC₅₀ values (1 to 3 magnitude lower) for CDK 1, CDK 2, CDK 7, CDK 9, CDK 16, and CDK 17, than Comparator-1 and Comparator-2.

A method disclosed herein can be used to treat, for example, an infectious disease, a proliferative disease, a cancer, a solid tumor, or a liquid tumor. Non-limiting examples of tumors that are treatable by a combination of a compound described herein can include solid tumors, solid tumors that are refractory to prior treatment with conventional chemotherapy, and solid tumors that respond to initial chemotherapy but subsequently relapsed. In some embodiments, the tumor is an ovarian cancer tumor.

A tumor response due to a method disclosed herein can be measured based on the Response Evaluation Criteria in Solid Tumors (RECIST) classification of responses. To use RECIST, there must be at least one tumor that can be measured on x-rays. CT scans, or MRI scans. RECIST assigns four categories of response: complete response (CR), a partial response (PR), progressive disease (PD), and stable disease (SD). Key features of the RECIST include definitions of minimum size of measurable lesions, instructions on how many lesions to follow, and the use of unidimensional, rather than bidimensional, measures for overall evaluation of tumor burden. Tumor measurement can be assessed with RECIST every 4 weeks, 5 weeks, 6 weeks, 7 weeks, 8 weeks, 9 weeks, 10 weeks, 11 weeks, 12 weeks, 13 weeks, or 14 weeks.

In some embodiments, the present disclosure provides a method of treating ovarian cancer, e.g., serous ovarian cancer, e.g., low grade serous ovarian cancer in a subject in need thereof, the method comprising administering to the subject a therapeutically-effective amount of a compound of formula (I), or a pharmaceutically-acceptable salt thereof. In some embodiments, the present disclosure provides a method of treating ovarian cancer, e.g., serous ovarian cancer, e.g., low grade serous ovarian cancer in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile or a pharmaceutically-acceptable salt thereof. In some embodiments, the present disclosure provides a method of treating ovarian cancer, e.g., serous ovarian cancer, e.g., low grade serous ovarian cancer in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile monolactate.

In some embodiments, the ovarian cancer is high grade serous ovarian carcinoma (HGSOC). High grade serous ovarian carcinoma originates from a serous epithelial layer in the abdominopelvic cavity and is mainly found in the ovary. In some embodiments, the high grade serous ovarian carcinoma originates from a fallopian tube epithelium. In some embodiments, the high grade serous ovarian carcinoma originates from an extra-uterine Müllerian epithelium. In some embodiments, the high grade serous ovarian carcinoma originates from ovarian tissue. In some embodiments, the high grade serous ovarian carcinoma originates from a Coelomic epithelium. In some embodiments, the high grade serous ovarian carcinoma is classified as immunoreactive. In some embodiments, the high grade serous carcinoma is classified as proliferative. In some embodiments, the high grade serous ovarian carcinoma is classified as differentiated. In some embodiments, the high grade serous ovarian carcinoma is classified as mesenchymal.

In some embodiments, the present disclosure provides a method of treating high grade serous ovarian carcinoma in a subject in need thereof, the method comprising administering to the subject a therapeutically-effective amount of a compound of formula (I), or a pharmaceutically-acceptable salt thereof. In some embodiments, the present disclosure provides a method of treating high grade serous ovarian carcinoma in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile or a pharmaceutically-acceptable salt thereof. In some embodiments, the present disclosure provides a method of treating high grade serous ovarian carcinoma in a subject in need thereof, the method comprising administering to the subject a therapeutically-effective amount of 8-cyclopentyl-2-((4-(4-methylpiperazin-1 yl)phenyl)amino)-7-oxo-7.8-dihydropyrido[2,3-d]pyrimidine-6-carbonitrile monolactate.

In some embodiments, the ovarian cancer is low grade serous ovarian carcinoma (LGSOC). In some embodiments, the low grade serous ovarian carcinoma is ovarian carcinoma. In some embodiments, the low grade serous ovarian carcinoma is fallopian tube carcinoma. In some embodiments, the low grade serous ovarian carcinoma is primary peritoneal carcinoma (PPC). In some embodiments, the low grade serous ovarian carcinoma is progesterone-receptor positive. In some embodiments, the low grade serous ovarian carcinoma is estrogen-receptor positive and progesterone-receptor positive. In some embodiments, the low grade serous ovarian carcinoma is progesterone positive. In some embodiments, the low grade serous ovarian carcinoma is estrogen positive, progesterone positive, and hormone-receptor positive.

In some embodiments, the present disclosure provides a method of treating low grade serous ovarian carcinoma in a subject in need thereof, the method comprising administering to the subject a therapeutically-effective amount of a compound of formula (I), or a pharmaceutically-acceptable salt thereof. In some embodiments, the present disclosure provides a method of treating low grade serous ovarian carcinoma in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile or a pharmaceutically-acceptable salt thereof. In some embodiments, the present disclosure provides a method of treating low grade serous ovarian carcinoma in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile monolactate.

In some embodiments, the low grade serous ovarian carcinoma is ovarian carcinoma. In some embodiments, the present disclosure provides a method of treating ovarian carcinoma in a subject in need thereof, the method comprising administering to the subject a therapeutically-effective amount of a compound of formula (I), or a pharmaceutically-acceptable salt thereof. In some embodiments, the present disclosure provides a method of treating ovarian carcinoma in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile or a pharmaceutically-acceptable salt thereof. In some embodiments, the present disclosure provides a method of treating ovarian carcinoma in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile monolactate.

In some embodiments, the low grade serous ovarian carcinoma is fallopian tube carcinoma. In some embodiments, the present disclosure provides a method of treating fallopian tube carcinoma in a subject in need thereof, the method comprising administering to the subject a therapeutically-effective amount of a compound of formula (I), or a pharmaceutically-acceptable salt thereof. In some embodiments, the present disclosure provides a method of treating fallopian tube carcinoma in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile or a pharmaceutically-acceptable salt thereof. In some embodiments, the present disclosure provides a method of treating fallopian tube carcinoma in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile monolactate.

In some embodiments, the low grade serous ovarian carcinoma is primary peritoneal carcinoma (PPC). In some embodiments, the present disclosure provides a method of treating primary peritoneal carcinoma in a subject in need thereof, the method comprising administering to the subject a therapeutically-effective amount of a compound of formula (I), or a pharmaceutically-acceptable salt thereof. In some embodiments, the present disclosure provides a method of treating primary peritoneal carcinoma in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile or a pharmaceutically-acceptable salt thereof. In some embodiments, the present disclosure provides a method of treating primary peritoneal carcinoma in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile monolactate.

In some embodiments, the administering comprises a second line of therapy. In some embodiments, the subject received a therapy other than the compound for the ovarian cancer (e.g., the high grade serous ovarian carcinoma or the low grade serous ovarian carcinoma) prior to the administering. In some embodiments, the therapy was received after the subject was diagnosed with the ovarian cancer, e.g., serous ovarian cancer, e.g., low grade serous ovarian cancer. In some embodiments, the therapy was received after the subject was diagnosed with high grade serous ovarian carcinoma. In some embodiments, the therapy was received after the subject was diagnosed with low grade serous ovarian carcinoma. In some embodiments, the therapy was received after the subject was diagnosed with ovarian carcinoma. In some embodiments, the therapy was received after the subject was diagnosed with fallopian tube carcinoma. In some embodiments, the therapy was received after the subject was diagnosed with primary peritoneal carcinoma. In some embodiments, the subject did not respond to the therapy. In some embodiments, the subject experienced a relapse of the ovarian cancer, e.g., serous ovarian cancer, e.g., low grade serous ovarian cancer after the therapy. In some embodiments, the subject experienced a relapse of high grade serous ovarian carcinoma after the therapy. In some embodiments, the subject experienced a relapse of low grade serous ovarian carcinoma after the therapy. In some embodiments, the subject experienced a relapse of the ovarian carcinoma after the therapy. In some embodiments, the subject experienced a relapse of the fallopian tube carcinoma after the therapy. In some embodiments, the subject experienced a relapse of primary peritoneal carcinoma after the therapy.

### Combination Therapy

The present disclosure also provides a combination and method for using such of a compound disclosed herein, for example, 8-cyclopentyl-2-((4-(4-methylpiperazin-1 yl)phenyl)amino)-7-oxo-7,8-dihydropyrido[2,3-d]pyrimidine-6-carbonitrile or a pharmaceutically-acceptable salt thereof, with an estrogen blocker and/or an aromatase inhibitor, for example letrozole.

In some embodiments, a compound of the disclosure, e.g., a compound of formula (I), such as 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof, is administered in combination with a second compound, e.g., a drug. In some embodiments, a compound of the disclosure, e.g., a compound of formula (I), such as 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof, is administered in combination with an estrogen receptor modulator. In some embodiments, a compound of the disclosure, e.g., a compound of formula (I), such as 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof, is administered in combination with an estrogen receptor blocker, e.g., an aromatase inhibitor such as letrozole. In some embodiments, a compound of the disclosure, e.g., a compound of formula (I), such as 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof, is administered in combination with a progestin such as megestrol or esters thereof (e.g., megestrol acetate). In some embodiments, a compound of the disclosure, e.g., a compound of formula (I), such as 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof, is administered in combination with an estrogen receptor degrader. In some embodiments, a compound of the disclosure, e.g., a compound of formula (I), such as 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof, is administered in combination with an angiogenesis inhibitor. In some embodiments, a compound of the disclosure, e.g., a compound of formula (I), such as 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof, is administered in combination with a vascular endothelial growth factor (VEGF) inhibitor. In some embodiments, a compound of the disclosure, e.g., a compound of formula (I), such as 8-cyclopentyl-2-((4-(4-methylpiperazin-1-yl)phenyl)amino)-7-oxo-7,8-dihydropyrido[ 2,3-d]pyrimidine-6-carbonitrile or a pharmaceutically-acceptable salt thereof, is administered in combination with a phosphoinositide 3-kinase (PI3K) inhibitor. In some embodiments, after administration of the combination therapy described herein, there is transition to maintenance estrogen blocker and/or an aromatase inhibitor, for example letrozole.

### Estrogen Receptor Blockers

Estrogens stimulate or maintain the growth of some cancers (e.g., ovarian cancer, e.g., serous ovarian cancer, e.g., low grade serous ovarian cancer). Treatment of ovarian cancer thought to be hormonally responsive (e.g., estrogen and/or progesterone receptor positive) is aimed at decreasing estrogen levels or inhibiting estrogen effects. In some embodiments, these interventions lead to decreased tumor mass or delayed progression of tumor growth.

Estrogen blockers block the production of estrogens (e.g., estradiol) or prevent estrogens (e.g. estradiol) from mediating biological effects in the body. Estrogen blockers act by blocking the estrogen receptor and/or directly inhibiting or suppressing estrogen production. Estrogen blockers are divided into classes by whether the estrogen blockers reduce the production of estrogen (aromatase inhibitors and antigonadotropins) or whether the estrogen blockers reduce the response to estrogen (antiestrogens and estrogen antagonists). By producing less estrogen, or by blocking the response to estrogen, estrogen blockers slow or inhibit the growth of cancer cells that require estrogen to stimulate growth (e.g., ovarian cells, endometrial cells, uterine cells). Non-limiting examples of estrogen blockers include aromatase inhibitors, e.g., letrozole (Femara), anastrozole (Arimidex), exemestane (Aromasin), tamoxifen, testolactone (Teslac), ethamoxytriphetol, clomifene, and raloxifene. Non-limiting examples of estrogen blockers include selective estrogen modulator (SERM), e.g., tamoxifen, toremifene, and raloxifene. Non-limiting examples of estrogen blockers include selective estrogen receptor degrader (SERD), e.g., fulvestrant and elacestrant.

Non-limiting examples of cells whose activity can be modulated by a combination therapy of a compound described herein and an estrogen blocker include secretory cells, cells with cilia, basal cells, red blood cells, mesenchymal cells, pluripotential mesenchymal cells, predecidual cells, epithelial cells, histiocytes, granulocytes, glandular cells, stromal cells, endometrial cells, follicular cells, ovarian surface epithelial cells, and theca cells.

Non-limiting examples of tumors that are treatable by a combination of a compound described herein and an estrogen blocker include solid tumors, solid tumors that are refractory to prior treatment with conventional chemotherapy, and solid tumors that respond to initial chemotherapy and subsequently relapse, as evidenced, e.g., by disease progression.

Non-limiting examples of cancers that are treatable by a combination of a compound described herein and an estrogen blocker include ovarian cancer, high grade serous ovarian carcinoma, low grade serous ovarian carcinoma, ovarian carcinoma, fallopian tube carcinoma, primary peritoneal carcinoma, ovarian cancer that is refractory to prior treatment with conventional chemotherapy, and ovarian cancer that responds to initial chemotherapy but subsequently relapses.

### Aromatase Inhibitors

In postmenopausal women, estrogens are mainly derived from the action of the aromatase enzyme. This enzyme converts adrenal androgens (e.g., androstenedione and testosterone) to estrone and estradiol. The suppression of estrogen biosynthesis in peripheral tissues and in the cancer tissue is achieved by inhibiting the aromatase enzyme.

Letrozole is a nonsteroidal competitive inhibitor of the aromatase enzyme system. Letrozole inhibits the conversion of androgens to estrogens. Letrozole selectively inhibits gonadal steroidogenesis but has no significant effect on adrenal mineralocorticoid or glucocorticoid synthesis. Letrozole inhibits the aromatase enzyme by competitively binding to the heme of the cytochrome P450 subunit of the enzyme, resulting in a reduction of estrogen biosynthesis. Treatment of women with letrozole significantly lowers serum estrone, estradiol and estrone sulfate.

The structure of letrozole is depicted below.

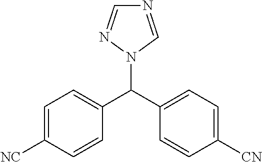 [SMILES: N#Cc1ccc(C(c2ccc(C#N)cc2)n2cncn2)cc1]

4,4′-((1H-1,2,4-triazol-1-yl)methylene)dibenzonitrile. Pharmaceutically-acceptable salts of 4,4′-((1H-1,2,4-triazol-1-yl)methylene)dibenzonitrile can also be used.
Non-limiting examples of aromatase inhibitor include anastrozole (Arimidex), Exemestane (Aromasin) and Letrozole (Femara).

### Progestins

Progestins are synthetic forms of the naturally-occurring hormone progesterone. Progestins counteract estrogen effects on the body and function similarly to estrogen blockers. Non-limiting examples of progestins include megestrol acetate, acetomepregenol, chlormadinone acetate, cyproterone acetate, danazol, drospirenone, gestrinone, levonorgestrel, medrogestone, norethisterone, norethisterone acetate, norgestrel, oxendolone, osaterone acetate, trimegestone, norethindrone, norethindrone acetate, norethynodrel, ethynodiol diacetate, a third desogestrel, gestodene, norgestimate, dienogest, nestorone, and nomegestrol acetate.

Megestrol acetate is a synthetic derivative of progesterone. Megestrol binds to progesterone receptors and changes the hormone balance in the body, which may inhibit or stop cancers, e.g., ovarian cancer, e.g., serous ovarian cancer, e.g., low grade serous ovarian cancer associated with estrogen.

The structure of megestrol acetate is depicted below.

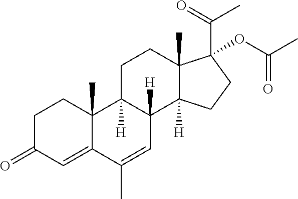

17-Hydroxy-6-methyl pregna-4,6-diene-3,20-dione acetate.

### Selective Estrogen Receptors Degraders

A selective estrogen receptor degrader or down-regulator (SERD) can bind to the estrogen receptor and cause the estrogen receptor to be degraded and thus downregulated. SERDs are used to treat estrogen receptor-sensitive or progesterone receptor-sensitive cancers, e.g., ovarian cancer, e.g., serous ovarian cancer, e.g., low grade serous ovarian cancer along with other similar classes of drugs such as estrogen blockers and aromatase inhibitors. Non-limiting examples of SERD include fulvestrant and elacestrant.

### Angiogenesis Inhibitors and Vascular Endothelial Growth Factor Inhibitors

Angiogenesis inhibitors interfere with blood vessel formation. Normally, the angiogenesis stimulating and inhibiting effects of these chemical signals are balanced so that blood vessels form only when and where vessels are needed. However, these signals can become unbalanced, causing increased blood vessel growth that can lead to abnormal conditions or disease, such as cancer, e.g., ovarian cancer, e.g., serous ovarian cancer, e.g., low grade serous ovarian cancer.

Vascular endothelial growth factors (VEGF) are angiogenic factors that are growth factors for vascular endothelial cells. When VEGF and other endothelial growth factors bind to receptors on endothelial cells, signals within these cells are initiated that promote the growth and survival of new blood vessels.

Angiogenesis inhibitors interfere with various steps in blood vessel growth. Some angiogenesis inhibitors are monoclonal antibodies that specifically recognize and bind to VEGF to block the binding of VEGF to the VEGF receptor. Other angiogenesis inhibitors bind to VEGF, the VEGFs receptor, other receptors on the surface of endothelial cells, or to other proteins in the downstream signaling pathways. Some angiogenesis inhibitors are immunomodulatory drugs-agents that stimulate or suppress the immune system—that also have antiangiogenic properties. Non-limiting examples of VEGF inhibitors include bevacizumab (Avastin), sorafenib (Nexavar), sunitinib (Sutent), nilotinib (Tasigna), pazopanib (Votrient), and dasatinib (Sprycel).

### PI3K Inhibitors

Phosphoinositide 3-kinase (PI3K) inhibitors inhibit one or more of the phosphoinositide 3-kinase enzymes. These enzymes form part of the PI3K/AKT/mTOR pathway, which is a pathway involved in cell growth and survival, and other processes that are frequently activated in many cancers, e.g., ovarian cancer, e.g., serous ovarian cancer, e.g., low grade serous ovarian cancer. By inhibiting these enzymes. PI3K inhibitors cause cell death, inhibit the proliferation of malignant cells, and interfere with several signaling pathways. PI3K inhibitors are usually given to treat certain cancers, e.g., ovarian cancer, e.g., serous ovarian cancer, e.g., low grade serous ovarian cancer, that have relapsed or are unresponsive to other cancer treatments. Non-limiting examples of PI3K inhibitors include alpelisib, copanlisib, duvelisib, and idelalisib.

### Administration of Compounds of the Disclosure

### Compound (1)

A compound disclosed herein, for example, 8-cyclopentyl-2-((4-(4-methylpiperazin-1-yl)phenyl)amino)-7-oxo-7,8-dihydropyrido[2,3-d]pyrimidine-6-carbonitrile (formula 1), can be formulated as a capsule. A capsule may be a hard capsule. A capsule may be a soft capsule. A capsule may be a soft gelatin capsule. In some embodiments, a compound disclosed herein can be formulated as a hard capsule, the hard capsule comprising an amount of 8-cyclopentyl-2-((4-(4-methylpiperazin-1-yl)phenyl)amino)-7-oxo-7,8-dihydropyrido[2,3-d]pyrimidine-6-carbonitrile monolactate equivalent to 40 mg of 8-cyclopentyl-2-((4-(4-methylpiperazin-1-yl)phenyl)amino)-7-oxo-7,8-dihydropyrido[2,3-d]pyrimidine-6-carbonitrile. Alternatively, a compound described herein can be formulated as a tablet.

In some embodiments, the present disclosure provides a pharmaceutical composition comprising, in a unit dosage form, an amount of 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof, equivalent to 40 mg of a compound described herein.

In some embodiments, the present disclosure provides a pharmaceutical composition comprising, in a unit dosage form, an amount of 8-cyclopentyl-2-((4-(4-methylpiperazin-1-yl)phenyl)amino)-7-oxo-7,8-dihydropyrido[2,3-d]pyrimidine-6-carbonitrile monolactate, equivalent to 40 mg of 8-cyclopentyl-2-((4-(4-methylpiperazin-1-yl)phenyl)amino)-7-oxo-7,8-dihydropyrido[ 2,3-d]pyrimidine-6-carbonitrile.

In some embodiments, the present disclosure provides a pharmaceutical composition comprising, in a unit dosage form, 48.4 mg of 8-cyclopentyl-2-((4-(4-methylpiperazin-1-yl)phenyl)amino)-7-oxo-7,8-dihydropyrido[ 2,3-d]pyrimidine-6-carbonitrile monolactate.

In some embodiments, 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt is administered in oral capsules, swallowed with water in the morning in a fasted state, at least 1 hour before ingesting food. In some embodiments, a morning dose is taken after an overnight fast an hour before ingesting food. In some embodiments, a compound described herein is administered every day. In some embodiments, a compound described herein is administered every day for 4 weeks. In some embodiments, a compound described herein is on a 4-week cycle of: (i) a continuous, three-week period of once-daily administration; and (ii) immediately following the three-week period, one week of no administration.

### Letrozole

A compound disclosed herein, for example, letrozole, can be formulated as tablet. In some embodiments, letrozole can be formulated as a tablet, the tablet comprising, in a unit dosage form, a therapeutically-effective amount of letrozole, and a pharmaceutically-acceptable excipient.

In some embodiments, letrozole is provided as 2.5 mg tablets for oral administration. The tablets can be colored, e.g., yellow; and can be uncoated or film-coated. Inactive ingredients may include colloidal silicon dioxide, ferric oxide, hydroxypropyl methylcellulose, lactose monohydrate, magnesium stearate, maize starch, microcrystalline cellulose, polyethylene glycol, sodium starch glycolate, talc, and titanium dioxide. In some embodiments, letrozole is given at a dose of one 2.5 mg tablet administered once a day without regards to food.

### Megestrol Acetate

A compound disclosed herein, for example, megestrol acetate, can be formulated as a suspension, for example a suspension suitable for oral administration. In some embodiments, megestrol acetate oral suspension contains 625 mg of megestrol acetate per 5 mL (125 mg/mL). In some embodiments, megestrol acetate oral suspension contains 800 mg per 20 mL (40 mg/mL). In some embodiments, megestrol acetate oral suspension contains one or more of the following inactive ingredients: alcohol (up to 0.06% v/v), lime flavor, citric acid monohydrate, docusate sodium, hydroxypropyl methylcellulose, natural and artificial lemon flavor, purified water, sodium benzoate, sodium citrate, dihydrate, and sucrose.

In some embodiments, megestrol acetate can be formulated as a tablet. In some embodiments, tablets contain 20 mg megestrol acetate. In some embodiments, tablets contain 40 mg megestrol acetate. In some embodiments, megestrol acetate tablets contain one or more of the following inactive ingredients: acacia spray dried, colloidal silicon dioxide, corn starch, di-calcium phosphate dihydrate powder, lactose hydrous impalpable, magnesium stearate and pregelatinized starch.

### Pharmaceutical Compositions

A pharmaceutical composition of the disclosure can be a combination of any pharmaceutical compounds described herein with other chemical components, such as carriers, stabilizers, diluents, dispersing agents, suspending agents, thickening agents, and/or excipients. The pharmaceutical composition facilitates administration of the compound to an organism. Pharmaceutical compositions can be administered in therapeutically-effective amounts as pharmaceutical compositions by various forms and routes including, for example, intravenous, subcutaneous, intramuscular, inhalation, oral, parenteral, ophthalmic, otic, subcutaneous, transdermal, nasal, intravitreal, intratracheal, intrapulmonary, transmucosal, vaginal, and topical administration.

Formulations can be modified depending upon the route of administration chosen. Pharmaceutical compositions comprising a compound described herein can be manufactured, for example, by mixing, dissolving, emulsifying, encapsulating, entrapping, or compression processes.

Non-limiting examples of dosage forms suitable for use in a method disclosed herein include feed, food, pellet, lozenge, liquid, elixir, aerosol, inhalant, spray, powder, tablet, pill, capsule, gel, geltab, nanosuspension, nanoparticle, microgel, suppository troches, aqueous or oily suspensions, ointment, patch, lotion, dentifrice, emulsion, creams, drops, dispersible powders or granules, emulsion in hard or soft gel capsules, syrups, phytoceuticals, nutraceuticals, and any combination thereof.

Pharmaceutical compositions can be formulated by combining the active compounds with pharmaceutically-acceptable carriers or excipients. Non-limiting examples of pharmaceutically-acceptable excipients suitable for use in the method disclosed herein include granulating agents, binding agents, lubricating agents, disintegrating agents, sweetening agents, glidants, anti-adherents, anti-static agents, surfactants, anti-oxidants, gums, coating agents, coloring agents, flavoring agents, coating agents, plasticizers, preservatives, suspending agents, emulsifying agents, anti-microbial agents, plant cellulosic material and spheronization agents, and any combination thereof.

Non-limiting examples of pharmaceutically-acceptable carriers include saline solution, Ringer's solution and dextrose solution. Further carriers include sustained release preparations such as semipermeable matrices of solid hydrophobic polymers containing the compound disclosed herein, where the matrices are in the form of shaped articles, such as films, liposomes, microparticles, and microcapsules.

For oral administration, pharmaceutical compositions can be formulated by combining the active compounds with pharmaceutically-acceptable carriers or excipients into a unit dosage form which can be solid or liquid. Non-limiting examples of oral solid forms include tablets, powders, pills, dragees, capsules, liquids, gels, syrups, elixirs, slurries, or suspensions for oral ingestion by a subject. Pharmaceutical preparations for oral use can be obtained by mixing one or more solid excipients with one or more compounds described herein, optionally grinding the resulting mixture, and processing the mixture of granules, after adding suitable auxiliaries, if desired, to obtain tablets or dragee cores. Cores can be provided with suitable coatings. For this purpose, concentrated sugar solutions can be used. The solutions can contain an excipient such as gum arabic, talc, polyvinylpyrrolidone, carbopol gel, polyethylene glycol, or titanium dioxide, lacquer solutions, and suitable organic solvents or solvent mixtures. Dyestuffs or pigments can be added to the tablets or dragee coatings, for example, for identification or to characterize different combinations of active compound doses.

Pharmaceutical preparations that can be used orally include push-fit capsules made of gelatin and soft, sealed capsules made of gelatin and a plasticizer, such as glycerol or sorbitol. Pharmaceutical preparations that can be used orally include coated and uncoated tablets. In some embodiments, the capsule comprises a hard gelatin capsule, the capsule comprising one or more of pharmaceutical, bovine, and plant gelatins. A gelatin can be alkaline-processed. The capsule or tablet can contain the active ingredients in admixture with filler such as lactose, binders such as starches, or lubricants such as talc or magnesium stearate, and stabilizers. In soft capsules, the active compounds can be dissolved or suspended in suitable liquids, such as fatty oils, liquid paraffin, or liquid polyethylene glycols. Stabilizers can be added. All formulations for oral administration are provided in dosages suitable for such administration.

For oral administration of a liquid unit dosage form, pharmaceutical compositions can be formulated by combining the active compounds with pharmaceutically-acceptable carriers or excipients. Such carriers can be used to formulate liquids, gels, syrups, elixirs, slurries, or suspensions, for oral ingestion by a subject. Non-limiting examples of solvents used in an oral dissolvable formulation can include water, ethanol, isopropanol, saline, physiological saline, DMSO, dimethylformamide, potassium phosphate buffer, phosphate buffer saline (PBS), sodium phosphate buffer, 4-2-hydroxyethyl-1-piperazineethanesulfonic acid buffer (HEPES), 3-(N-morpholino) propanesulfonic acid buffer (MOPS), piperazine-N,N′-bis(2-ethanesulfonic acid) buffer (PIPES), and saline sodium citrate buffer (SSC). Non-limiting examples of co-solvents used in an oral dissolvable formulation can include sucrose, urea, cremaphor, DMSO, and potassium phosphate buffer.

Parenteral injections can be formulated for bolus injection or continuous infusion. The pharmaceutical compositions can be in a form suitable for parenteral injection as a sterile suspension, solution or emulsion in oily or aqueous vehicles such as saline or water for injection, and can contain formulatory agents such as suspending, stabilizing and/or dispersing agents. Pharmaceutical formulations for parenteral administration include aqueous solutions of the active compounds in water-soluble form. Suspensions of the active compounds can be prepared as oily injection suspensions. Suitable lipophilic solvents or vehicles include fatty oils such as sesame oil, or synthetic fatty acid esters, such as ethyl oleate or triglycerides, or liposomes. The suspension can also contain suitable stabilizers or agents which increase the solubility of the compounds to allow for the preparation of highly concentrated solutions. Alternatively, the active ingredient can be in powder form for constitution with a suitable vehicle, e.g., sterile pyrogen-free water, before use.

The active compounds can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives. The compounds of the disclosure can be applied topically to the skin, or a body cavity, for example, oral, vaginal, bladder, cranial, spinal, thoracic, or pelvic cavity of a subject. The compounds of the disclosure can be applied to an accessible body cavity.

The compounds can also be formulated in rectal compositions such as enemas, rectal gels, rectal foams, rectal aerosols, suppositories, jelly suppositories, or retention enemas, containing conventional suppository bases such as cocoa butter or other glycerides, and synthetic polymers such as polyvinylpyrrolidone and PEG. In suppository forms of the compositions, a low-melting wax such as a mixture of fatty acid glycerides, optionally in combination with cocoa butter, can be used.

For buccal or sublingual administration, the compositions can be tablets, lozenges, or gels.

Formulations suitable for transdermal administration of the active compounds can employ transdermal delivery devices and transdermal delivery patches, and can be lipophilic emulsions or buffered aqueous solutions, dissolved or dispersed in a polymer or an adhesive. Such patches can be constructed for continuous, pulsatile, or on demand delivery of pharmaceutical compounds. Transdermal delivery can be accomplished by iontophoretic patches. Transdermal patches can provide controlled delivery. The rate of absorption can be slowed by using rate-controlling membranes or by trapping the compound within a polymer matrix or gel. Absorption enhancers can be used to increase absorption. An absorption enhancer or carrier can include absorbable pharmaceutically-acceptable solvents to assist passage through the skin. For example, transdermal devices can be in the form of a bandage comprising a backing member, a reservoir containing compounds and carriers, a rate controlling barrier to deliver the compounds to the skin of the subject at a controlled and predetermined rate over a prolonged period of time, and adhesives to secure the device to the skin or the eye.

For administration by inhalation, the active compounds can be in a form as an aerosol, a vapor, a mist, or a powder. Inhalation can occur through by nasal delivery, oral delivery, or both.

Nasal or intranasal administration involves insufflation of compounds through the nose, for example, nasal drops and nasal sprays. This route of administration can result in local and/or systemic effects. Inhaler or insufflator devices can be used for nose-to-lung delivery of compounds described herein.

A pharmaceutical composition can be administered in a local or systemic manner, for example, via injection of the compound directly into an organ, optionally in a depot or sustained release formulation or implant. Pharmaceutical compositions can be provided in the form of a rapid release formulation, in the form of an extended release formulation, or in the form of an intermediate release formulation. A rapid release form can provide an immediate release. An extended release formulation can provide a controlled release or a sustained delayed release.

In practicing the methods of treatment or use provided herein, therapeutically-effective amounts of the compounds described herein are administered in pharmaceutical compositions to a subject having a disease or condition to be treated. In some embodiments, the subject is a mammal such as a human. A therapeutically-effective amount can vary widely depending on the severity of the disease, the age and relative health of the subject, the potency of the compounds used, and other factors. The compounds can be used singly or in combination with one or more therapeutic agents as components of mixtures.

Methods for the preparation of compositions comprising the compounds described herein include formulating the compounds with one or more inert, pharmaceutically-acceptable excipients or carriers to form a solid, semi-solid, or liquid composition. Solid compositions include, for example, powders, tablets, dispersible granules, capsules, and cachets. Liquid compositions include, for example, solutions in which a compound is dissolved, emulsions comprising a compound, or a solution containing liposomes, micelles, or nanoparticles comprising a compound as disclosed herein. Semi-solid compositions include, for example, gels, suspensions and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Non-limiting examples of dosage forms suitable for use in the disclosure include liquid, powder, gel, nanosuspension, nanoparticle, microgel, aqueous or oily suspensions, emulsion, and any combination thereof.

Non-limiting examples of pharmaceutically-acceptable excipients suitable for use in the disclosure include binding agents, disintegrating agents, anti-adherents, anti-static agents, surfactants, anti-oxidants, coating agents, coloring agents, plasticizers, preservatives, suspending agents, emulsifying agents, anti-microbial agents, spheronization agents, solubilizers, stabilizers, tonicity enhancing agents, buffers and any combination thereof.

A composition of the disclosure can be, for example, an immediate release form or a controlled release formulation. An immediate release formulation can be formulated to allow the compounds to act rapidly. Non-limiting examples of immediate release formulations include readily dissolvable formulations. A controlled release formulation can be a pharmaceutical formulation that has been adapted such that release rates and release profiles of the active agent can be matched to physiological and chronotherapeutic requirements or, alternatively, has been formulated to effect release of an active agent at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, and granular masses.

In some, a controlled release formulation is a delayed release form. A delayed release form can be formulated to delay a compound's action for an extended period of time. A delayed release form can be formulated to delay the release of an effective dose of one or more compounds, for example, for about 4, about 8, about 12, about 16, or about 24 h.

A controlled release formulation can be a sustained release form. A sustained release form can be formulated to sustain, for example, the compound's action over an extended period of time. A sustained release form can be formulated to provide an effective dose of any compound described herein (e.g., provide a physiologically-effective blood profile) over about 4, about 8, about 12, about 16 or about 24 h.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980); and Pharmaceutical Dosage Forms and Drug Delivery Systems, Seventh Ed. (Lippincott Williams & Wilkins 1999), each of which is incorporated by reference in its entirety.

Subjects can be, for example, elderly adults, adults, adolescents, pre-adolescents, children, toddlers, infants, neonates, and non-human animals. In some embodiments, a subject is a patient.

A method disclosed herein relates to administering the compound disclosed herein as part of a pharmaceutical composition. In some embodiments, compositions of a compound disclosed herein can comprise a liquid comprising an active agent in solution, in suspension, or both. Liquid compositions can include gels. In some embodiments, the liquid composition is aqueous. Alternatively, the composition can be an ointment. In some embodiments, the composition is an in situ gellable aqueous composition. In some embodiments, the composition is an in situ gellable aqueous solution.

A pharmaceutically-acceptable excipient can be present in a pharmaceutical composition at a mass of between about 0.1% and about 99% by mass of the composition. For example, a pharmaceutically-acceptable excipient can be present in a pharmaceutical composition at a mass of between about 0.1% and about 95%, between about 0.1% and about 90%, between about 0.1% and about 85%, between about 0.1% and about 80%, between about 0.1% and about 75%, between about 0.1% and about 70%, between about 0.1% and about 65%, between about 0.1% and about 60%, between about 0.1% and about 55%, between about 0.1% and about 50%, between about 0.1% and about 45%, between about 0.1% and about 40%, between about 0.1% and about 35%, between about 0.1% and about 30%, between about 0.1% and about 25%, between about 0.1% and about 20%, between about 0.1% and about 15%, between about 0.1% and about 10%, between about 0.1% and about 5%, or between about 0.1% and about 1%, by mass of the formulation.

A pharmaceutically-acceptable excipient can be present at about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% by mass of the formulation.

### Dosing

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are packaged injectables, vials, or ampoules. Aqueous suspension compositions can be packaged in single-dose non-reclosable containers. Multiple-dose reclosable containers can be used, for example, in combination with or without a preservative. Formulations for parenteral injection can be presented in unit dosage form, for example, in ampoules, or in multi-dose containers with a preservative.

A compound described herein can be present in a composition in a range of from about 1 mg to about 5 mg, from about 5 mg to about 10 mg, from about 10 mg to about 15 mg, from about 15 mg to about 20 mg, from about 20 mg to about 25 mg, from about 25 mg to about 30 mg, from about 30 mg to about 35 mg, from about 35 mg to about 40 mg, from about 40 mg to about 45 mg, from about 45 mg to about 50 mg, from about 50 mg to about 55 mg, from about 55 mg to about 60 mg, from about 60 mg to about 65 mg, from about 65 mg to about 70 mg, from about 70 mg to about 75 mg, from about 75 mg to about 80 mg, from about 80 mg to about 85 mg, from about 85 mg to about 90 mg, from about 90 mg to about 95 mg, from about 95 mg to about 100 mg, from about 100 mg to about 125 mg, from about 125 mg to about 150 mg, from about 150 mg to about 175 mg, from about 175 mg to about 200 mg, from about 200 mg to about 225 mg, from about 225 mg to about 250 mg, from about 250 mg to about 300 mg, from about 300 mg to about 325 mg, from about 325 mg to about 350 mg, from about 350 mg to about 375 mg, from about 375 mg to about 400 mg, from about 400 mg to about 425, from about 425 mg to about 450 mg, from about 450) mg to about 475 mg, from about 475 mg to about 500 mg, from about 500 mg to about 525 mg, from about 525 mg to about 550 mg, from about 550) mg to about 575 mg, from about 575 mg to about 600 mg, from about 600 mg to about 625 mg, or from about 625 mg to about 650 mg.

A compound described herein can be present in a composition in an amount of about 5 mg, about 10 mg, about 15 mg, about 20 mg, about 25 mg, about 30 mg, about 35 mg, about 40) mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, or about 650 mg.

A compound described herein can be administered to a subject in an amount of about 0.1 mg/kg to about 500 mg/kg, about 1 mg/kg to about 500 mg/kg, about 0.1 mg/kg to about 300 mg/kg, about 1 mg/kg to about 300 mg/kg, or about 0.1 mg/kg to about 30 mg/kg. In some embodiments, the compound disclosed herein is administered to a subject in an amount of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 120 mg/kg, about 150 mg/kg, about 160 mg/kg, about 180 mg/kg, about 200 mg/kg, about 240 mg/kg, about 250 mg/kg, about 300 mg/kg, about 350 mg/kg, about 360 mg/kg, about 400 mg/kg, about 450 mg/kg, about 500 mg/kg, or about 600 mg/kg of the subject.

### Dosing Regimens

A dosing regimen disclosed herein can be, for example, once a day, twice a day, thrice a day, once a week, twice a week, or thrice a week. In some embodiments, a compound disclosed herein is administered once daily. In some embodiments, a compound disclosed herein is administered once daily for 28 days (one cycle). In some embodiments, a compound disclosed herein is administered once daily in one or more 28 day cycles. In some embodiments, a compound disclosed herein is administered in a four-week cycle of consecutive once daily administration for three weeks, followed by one week with no administrations.

In some embodiments, the present disclosure provides a method of treating ovarian cancer, e.g., serous ovarian cancer, e.g., low grade serous ovarian cancer in a subject in need thereof, the method comprising administering to the subject a therapeutically-effective amount of a compound of formula (I)

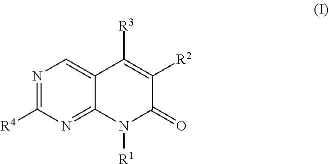

wherein:
   R¹ is alkyl, alkenyl, alkynyl, cycloalkyl, aryl, heteroaryl or heterocyclyl, each of which is unsubstituted or substituted, or hydrogen;
   R² is alkyl, alkenyl, alkynyl, cycloalkyl, aryl, heteroaryl, heterocyclyl, —CN, —C(O)R⁵, —C(O)OR⁵, —C(O)NR⁵R⁶, —C(═N)NR⁵R⁶, —OR⁵, —SR⁵, —NR′R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen;
   R³ is alkyl, alkenyl, alkynyl, cycloalkyl, aryl, heterocyclyl or heteroaryl, each of which is unsubstituted or substituted, or hydrogen or halogen;
   R⁴ is alkyl, alkenyl, alkynyl, cycloalkyl, aryl, heterocyclyl, heteroaryl, —CN, C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and each R⁵ and R⁶ is independently alkyl, alkenyl, alkynyl, cycloalkyl, aryl, heterocyclyl, or heteroaryl, each of which is independently substituted or unsubstituted, or hydrogen or halogen;
provided that, when R⁴ is —NR⁵R⁶ and one of R⁵ and R⁶ is pyridyl, then R² is not —C(O)CH₃,
or a pharmaceutically-acceptable salt thereof;
wherein the administering is once daily for at least 4 weeks.

In some embodiments, the present disclosure provides a method of treating ovarian cancer, e.g., serous ovarian cancer, e.g., low grade serous ovarian cancer in a subject in need thereof, the method comprising administering to the subject a therapeutically-effective amount of a compound of formula (I)

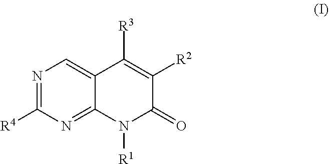

wherein:
   R¹ is alkyl, alkenyl, alkynyl, cycloalkyl, aryl, heteroaryl or heterocyclyl, each of which is unsubstituted or substituted, or hydrogen;
   R² is alkyl, alkenyl, alkynyl, cycloalkyl, aryl, heteroaryl, heterocyclyl, —CN, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen;
   R³ is alkyl, alkenyl, alkynyl, cycloalkyl, aryl, heterocyclyl or heteroaryl, each of which is unsubstituted or substituted, or hydrogen or halogen;
   R⁴ is alkyl, alkenyl, alkynyl, cycloalkyl, aryl, heterocyclyl, heteroaryl, —CN, C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
   each R⁵ and R⁶ is independently alkyl, alkenyl, alkynyl, cycloalkyl, aryl, heterocyclyl, or heteroaryl, each of which is independently substituted or unsubstituted, or hydrogen or halogen;
provided that, when R⁴ is —NR⁵R⁶ and one of R⁵ and R⁶ is pyridyl, then R² is not —C(O)CH₃,
or a pharmaceutically-acceptable salt thereof;
wherein the administering is a 4-week cycle of: (i) a continuous, three-week period of once-daily administration; and ii) immediately following the three-week period, one week of no administration.

A compound described herein can be administered before, during, or after the occurrence of a disease or condition, and the timing of administering the composition containing a compound can vary. For example, a compound can be used as a prophylactic and can be administered continuously to subjects with a propensity to conditions or diseases to lessen or reduce a likelihood of the occurrence of the disease or condition. A compound and composition can be administered to a subject during or as soon as possible after the onset of the symptoms. The administration of a compound can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein.

A compound can be administered as soon as is practical after the onset of a disease or condition is detected or suspected, and for a length of time necessary for the treatment of the disease, such as, for example, from about 1 month to about 3 months. In some embodiments, the length of time a compound can be administered can be about 1 day, about 2 days, about 3 days, about 4 days, about 5 days, about 6 days, about 1 week, about 2 weeks, about 3 weeks, about 4 weeks, about 1 month, about 5 weeks, about 6 weeks, about 7 weeks, about 8 weeks, about 2 months, about 9 weeks, about 10 weeks, about 11 weeks, about 12 weeks, about 3 months, about 13 weeks, about 14 weeks, about 15 weeks, about 16 weeks, about 4 months, about 17 weeks, about 18 weeks, about 19 weeks, about 20 weeks, about 5 months, about 21 weeks, about 22 weeks, about 23 weeks, about 24 weeks, about 6 months, about 7 months, about 8 months, about 9) months, about 10 months, about 11 months, about 1 year, about 13 months, about 14 months, about 15 months, about 16 months, about 17 months, about 18 months, about 19 months, about 20 months, about 21 months, about 22 months about 23 months, about 2 years, about 2.5 years, about 3 years, about 3.5 years, about 4 years, about 4.5 years, about 5 years, about 6 years, about 7 years, about 8 years, about 9 years, about 10 years, about 11 years, about 12 years, about 13 years, about 14 years, about 15 years, about 16 years, about 17 years, about 18 years, about 19 years, about 20 years, about 21 years, about 22 years, about 23 years, about 24 years, or about 25 years. The length of treatment can vary for each subject.

A dosing schedule for administration of a compound described herein can be consistent for the length of the dosing regimen. For example, a compound can be administered daily. Alternatively, or in addition to, a dosing schedule for administration of a compound described herein can include portions of time where dosing is paused. For example, a compound can be administered every day for 3 weeks and then not be administered for one week.

A dosing schedule for administration of a compound described herein can include once daily (QD), twice daily (BID), three times daily (TID), four times daily (QID), once weekly, twice weekly, three times weekly, once monthly, twice monthly, and once every other month. For example, a daily dose can be given in a single dose or divided into multiple doses to be administered in intervals, e.g., twice daily, three times daily, and the like. For example, a daily dose of 100 mg can be given, for example, once daily (100 mg), twice daily (50 mg per dose). In some embodiments, a dosing schedule for administration of a compound described herein is twice monthly (e.g., taken day 1 and day 15), followed by day 1 of subsequent cycles

Multiple therapeutic agents can be administered in any order or simultaneously. In some embodiments, a compound of the disclosure is administered in combination with, before, or after treatment with another therapeutic agent, e.g., a drug, such as an aromatase inhibitor. In some embodiments, a compound of the disclosure is administered at regular intervals, such as, for example, once daily, twice daily, thrice daily, etc. and the second therapeutic agent is administered daily or intermittently or on an as-needed basis. If simultaneously, the multiple therapeutic agents can be provided in a single, unified form, or in multiple forms, for example, as multiple separate unit dosage forms. The agents can be packed together or separately, in a single package or in a plurality of packages. One or all the therapeutic agents can be given in multiple doses. If not simultaneous, the timing between the multiple doses can vary to as much as about a month.

### Compound 1 Dosing Regimens

A dosing regimen disclosed herein can be, for example, one dose of 40 mg, one dose of 80 mg, one dose of 120 mg; one dose of 160 mg, or one dose of 200 mg of oral 8-cyclopentyl-2-((4-(4-methylpiperazin-1-yl)phenyl)amino)-7-oxo-7,8-dihydropyrido[2,3-d]pyrimidine-6-carbonitrile per day. Alternatively, the dosing regimen disclosed herein can be, for example, 40 mg twice daily, 60 mg twice daily, 80 mg twice daily, or 100 mg twice daily. In some embodiments, the dosing is oral.

In some embodiments, 8-cyclopentyl-2-((4-(4-methylpiperazin-1-yl)phenyl)amino)-7-oxo-7,8-dihydropyrido[2,3-d]pyrimidine-6-carbonitrile is administered once daily. In some embodiments, 8-cyclopentyl-2-((4-(4-methylpiperazin-1-yl)phenyl)amino)-7-oxo-7,8-dihydropyrido[ 2,3-d]pyrimidine-6-carbonitrile is administered once daily for 28 days (one cycle). In some embodiments, 8-cyclopentyl-2-((4-(4-methylpiperazin-1-yl)phenyl)amino)-7-oxo-7,8-dihydropyrido[2,3-d]pyrimidine-6-carbonitrile is administered once daily in one or more 28 day cycles. In some embodiments, 8-cyclopentyl-2-((4-(4-methylpiperazin-1-yl)phenyl)amino)-7-oxo-7,8-dihydropyrido[2,3-d]pyrimidine-6-carbonitrile is administered in a four-week cycle of consecutive once daily administration for three weeks, followed by one week with no administrations.

In some embodiments, the present disclosure provides a method of treating ovarian cancer, e.g., serous ovarian cancer, e.g., low grade serous ovarian cancer in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile or a pharmaceutically-acceptable salt thereof, wherein the administering is once daily for at least 4 weeks.

In some embodiments, the present disclosure provides a method of ovarian cancer e.g., serous ovarian cancer, e.g., low grade serous ovarian cancer in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile or a pharmaceutically-acceptable salt thereof, wherein the administering is a 4-week cycle of: (i) a continuous, three-week period of once-daily administration; and ii) immediately following the three-week period, one week of no administration.

In some embodiments, the present disclosure provides a method of treating ovarian cancer, e.g., serous ovarian cancer, e.g., low grade serous ovarian cancer in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile monolactate, wherein the administering is once daily for at least 4 weeks.

In some embodiments, the present disclosure provides a method of treating ovarian cancer e.g., serous ovarian cancer, e.g., low grade serous ovarian cancer in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile monolactate, wherein the administering is a 4-week cycle of: (i) a continuous, three-week period of once-daily administration; and ii) immediately following the three-week period, one week of no administration.

### Letrozole Dosing Regimens

A dosing regimen disclosed herein can be, for example, once a day, twice a day, thrice a day, once a week, twice a week, or thrice a week. In some embodiment, the dosing is oral. In some embodiments, a suitable amount letrozole can range from about 0.1 mg to about 100 mg per day, for example about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 2.5 mg, about 3 mg, about 3.5 mg, about 4 mg, about 4.5 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, or about 50 mg per day.

### EXAMPLES

### Example 1: Study to Evaluate an Oral Pharmaceutical Composition Disclosed Herein for Treating a Disease in a Subject

Summary: This study is a dose escalation study to investigate the safety, tolerability, and PK characteristics of 8-cyclopentyl-2-((4-(4-methylpiperazin-1-yl)phenyl)amino)-7-oxo-7,8-dihydropyrido[2,3-d]pyrimidine-6-carbonitrile (Compound 1) in patients with advanced cancers who have received and failed at least one prior treatment. The primary objective of this study is to assess the safety and tolerability of repeated daily dosing of Compound 1 in patients with relapsed and/or refractory advanced cancers. The secondary objective of this study is to establish a maximum tolerated dose (MTD) and a recommend phase 2 dose (RP2D) of orally administered Compound 1. In addition, the study explores efficacy of Compound 1 in cancer patients.

Study Design: This study is a dose finding study using 3+3 design for dose escalation. Three to six patients are enrolled per dose cohort, followed by up to 12 additional patients at the RP2D. Approximately 36 patients with advanced cancers are enrolled in the study, based on 4 dose levels and an expansion cohort. If additional dose escalations are required to establish the MTD/RP2D, then 3-6 additional patients are added per dose level.

Compound 1 is given in the form of a hard capsule comprising 48.4 mg 8-cyclopentyl-2-((4-(4-methylpiperazin-1-yl)phenyl)amino)-7-oxo-7,8-dihydropyrido[2,3-d]pyrimidine-6-carbonitrilemonolactate, equivalent to 40 mg of 8-cyclopentyl-2-((4-(4-methylpiperazin-1-yl)phenyl)amino)-7-oxo-7,8-dihydropyrido[2,3-d]pyrimidine-6-carbonitrile. The initial dose is 40 mg (one capsule) taken once daily for 28 days (one cycle). The dose is taken in the morning, on an empty stomach. Dose increments in the dose escalation 3+3 study are 40 mg of Compound 1 per cycle. Dose levels are 40 mg, 80 mg, 120 mg, 160 mg, etc., until a RP2D/MTD is reached. Each of the first three patients in the first and subsequent cohorts is assessed for dose limiting toxicities (DLT) during the first 28 days of treatment. If no patients experience a DLT, then enrollment to the next cohort begins at the next dose level. If one patient of the first three patients in a cohort experiences a DLT in the first 28 days, then an additional three patients are enrolled to that cohort for a total of six patients. If only one of the six patients in the cohort experiences a DLT in the first 28 days, then enrollment to the next cohort begins at the next dose level. If at any time two or more patients in a cohort experience a DLT in the first 28 days, the cohort is closed. The dose of that cohort is considered ‘non-tolerable’ and the prior dose level is defined as the MTD. Patients continue in the study until disease progression or intolerance or a decision to discontinue is reached.

### Study Objectives and Endpoints

The primary objective of this study is to assess the safety and tolerability of repeated daily dosing of Compound 1 in patients with relapsed and/or refractory advanced cancers. The primary endpoints include (DLTs, adverse events (AEs), deaths and other serious AEs.

The secondary objectives of this study are to establish a MTD of Compound land a RP2D of orally administered Compound land to characterize pharmacokinetics of Compound 1 following oral administration in patients with relapsed and/or refractory advanced cancers. Secondary endpoints include maximum plasma concentration (Cmax), area under the plasma concentration time curve (AUC), and half-life (t½).

Exploratory objectives of this study are to assess the efficacy of compound (1), by objective responses per RECIST, wherever appropriate for applicable tumors. Assessment of non-Hodgkin's Lymphoma and CNS tumors is by imaging techniques (CT. PET. MRI).

### Pharmacokinetics

Blood samples are collected pre- and post-dose on days 1 and 8 of the first cycle and pre-dose on day 1 of cycles 2 and 3 for pharmacokinetic (PK) analysis. Compound 1 concentrations are determined in plasma samples by a validated liquid chromatography-tandem mass spectrometry (LC-MS/MS) assay. Levels of Compound are determined at specified time points in the PK profile.

The following PK parameters are derived using model-independent analysis: time to reach Cmax (Tmax). Cmax, t½. AUC0-t. AUC0-α, CL, and Vss. Descriptive statistics (mean, median, range, standard deviation) for these parameters are provided and summarized by each dose group.

Cmax and Tmax are determined from the plasma concentration-time profile, and t½β is calculated as 0.693/k (where k is the terminal elimination rate constant, calculated by log-linear regression of the terminal portion of the concentration-time profile). AUC0-t is calculated by the linear trapezoidal rule and extrapolated to infinity using k to obtain AUC0-∞.

Pharmacokinetic parameters are calculated from Compound 1 concentration-time data using standard non-compartmental methods as implemented in WinNonlin. The maximum plasma concentration (Cmax) and time to reach Cmax (Tmax) are the observed values. The area under the plasma concentration-time curve (AUC) value is calculated to the last quantifiable sample (AUClast) by use of the linear trapezoidal rule. The AUC values are extrapolated to infinity (AUCinf) by dividing the last quantifiable concentration by the terminal disposition rate constant (λz), which is determined from the slope of the terminal phase of the concentration-time profile. The terminal half-life (T½) is calculated as 0.693 divided by λz. The apparent oral clearance (Cl/F) is calculated by dividing the dose administered by AUCinf.4. Pharmacokinetic data are analyzed by cohort.

Efficacy analysis. The efficacy variable is best overall response (ORR), using RECIST criteria, version 1.1. Objective tumor response is tabulated and summarized by the primary tumor type. If warranted, additional efficacy endpoints, like duration of response or time to progression, are analyzed

### Example 2: Study to Evaluate PK PD Results for a Pharmaceutical Composition Disclosed Herein for Treating a Disease in a Subject

Summary: This study is a dose escalation study to investigate the safety, tolerability, and PK characteristics of in-patients with advanced solid tumors who have received and failed at least one prior treatment. The primary objective of this study is to assess the safety and tolerability of repeated daily dosing of 8-cyclopentyl-2-((4-(4-methylpiperazin-1-yl)phenyl)amino)-7-oxo-7,8-dihydropyrido[2,3-d]pyrimidine-6-carbonitrile (Compound 1) in patients with relapsed and/or refractory advanced cancers. The secondary objective of this study is to establish a MT of Compound land RP2D of orally administered Compound 1. In addition, the study explores efficacy of Compound 1 in cancer patients.

Study design: The study includes a treatment period (1 year) and a follow-up period (90) days after the last dose). Subjects are pathologically confirmed to have malignant solid tumors, or advanced (metastatic or unresectable) malignant solid tumors and have previously failed standard treatment (e.g., targeted therapy, chemotherapy, biotherapy, immunotherapy, etc.), as evidenced by disease progression or intolerance toxicity. Subjects can also be included in the study if there are currently no effective treatments for cancer.

The study is divided into two stages, including a dosage escalation and a dose expansion cohort. The first phase is a dose escalation, using 3+3 design to determine MTD and/or RP2D. Three to six patients are enrolled per dose cohort, followed by up to 12 additional patients at the RP2D. Approximately 9-30 patients are enrolled in the first phase. If additional dose escalations are required to establish the MTD/RP2D, then 3-6 additional patients are added per dose level.

Patients receive study drug orally under fasted conditions before breakfast. Compound 1 is administered once daily continuously for 3 weeks with one week break. Safety, tolerability, and dose-limiting toxicity are evaluated after 4 weeks (28 days) of dosing.

Compound 1 is given in the form of a hard capsule comprising 48.4 mg 8-cyclopentyl-2-((4-(4-methylpiperazin-1-yl)phenyl)amino)-7-oxo-7,8-dihydropyrido[ 2,3-d]pyrimidine-6-carbonitrilemonolactate salt, equivalent to 40 mg of 8-cyclopentyl-2-((4-(4-methylpiperazin-1-yl)phenyl)amino)-7-oxo-7,8-dihydropyrido[2,3-d]pyrimidine-6-carbonitrile. The dose (one capsule) is taken in the morning, on an empty stomach. Dose increments in the dose escalation 3+3 study are 40 mg of Compound 1 per cycle. Dose levels are 40 mg, 80 mg, 120 mg, 160 mg, and 200 mg, or until a RP2D/MTD is reached. The highest escalation dose in the study is set at 200 mg. Dose escalation is performed as described in Example 1.

The second stage of the study is a dose expansion stage. The dose expansion stage enrolls 9-12 cancer patients (primarily advanced breast cancer and non-small cell lung cancer patients with HR(+) and HER2(−)). Test procedures are the same as in the dose expansion phase.

### Study Objectives and Endpoints

The primary objective of this study is to evaluate the tolerance, safety, and the anti-tumor efficacy of Compound 1 in patients having advanced solid tumors.

The secondary objective of this study is to characterize pharmacokinetics of Compound 1 following oral administration of single and multiple doses of Compound 1 in patients with relapsed and/or refractory advanced cancer. Secondary endpoints include maximum plasma concentration (Cmax), area under the plasma concentration time curve (AUC), and half-life (t½). The study evaluates the efficacy of Compound 1 in patients with solid tumors, including objective response rate ORR, progression-free survival PFS, duration of remission DOR, disease control rate DCR, etc.

### Pharmacokinetics

Blood samples are collected pre- and post-dose on days 1 and 8 of the first cycle and pre-dose on day 4 of the first cycle for pharmacokinetic (PK) analysis. Compound 1 PK is determined as described in Example 1.

Efficacy analysis. The efficacy variable of this study is best overall response (ORR), using RECIST criteria, version 1.1. Efficacy analysis includes: (1) Objective Remission Rate (ORR), defined as the proportion of subjects with complete remission (CR) and partial remission (PR) after treatment. (2) Disease Control Rate (DCR), defined as the proportion of subjects with complete remission (CR), partial remission (PR), and disease stabilization (SD) after treatment. (3) Time to remission (DOR), defined as the time from the initial recording of objective remission to the first occurrence of tumor progression, or death from any cause. And (4) Progression free survival (PFS), defined as treatment from initiation to tumor progression or death from any cause.

### Example 3: Dosing Regimen to Evaluate an Oral Pharmaceutical Composition Disclosed Herein with an Aromatase Inhibitor in a Subject

The study is a randomized, double-blind, placebo-controlled, study of 8-cyclopentyl-2-((4-(4-methylpiperazin-1-yl)phenyl)amino)-7-oxo-7,8-dihydropyrido[2,3-d]pyrimidine-6-carbonitrile (Compound 1)) in combination with an aromatase inhibitor (e.g., letrozole) versus placebo in combination with an aromatase inhibitor (e.g., letrozole) for patients with estrogen receptor positive advanced or recurrent ovarian cancer.

Study Design: This study is a treatment response study using 1:1 randomized double-blind study. Patients with ovarian cancer are randomized into one of two treatment arms: Arm A: (placebo): letrozole-placebo combination therapy; and Arm B (experimental): letrozole-Compound 1 combination therapy. Each cycle in the study is 28 days of treatment with tumors assessed every 12 weeks.

Compound 1 is given in the form of a hard capsule comprising 48.4 mg 8-cyclopentyl-2-((4-(4-methylpiperazin-1-yl)phenyl)amino)-7-oxo-7,8-dihydropyrido[2,3-d]pyrimidine-6-carbonitrilemonolactate salt, equivalent to 40 mg of 8-cyclopentyl-2-((4-(4-methylpiperazin-1-yl)phenyl)amino)-7-oxo-7,8-dihydropyrido[2,3-d]pyrimidine-6-carbonitrile. Compound 1 is taken once daily for 28 days (one cycle). Alternatively, Compound 1 is taken once daily continuously for 3 weeks with one week of no administration for a total of 28 days. Treatment continues until progression of disease or unacceptable toxicity. Compound 1 is taken in the morning, on an empty stomach.

Letrozole is given in the form of a 2.5 mg tablet. One 2.5 mg letrozole tablet is taken once on days 1-28 (one cycle). Treatment continues until progression of disease or unacceptable toxicity. Letrozole is taken in the morning, at the same time as either Compound 1 or the placebo.

Primary outcome measures Primary outcome measures are increase in Progression-Free Survival (PFS) in experimental arm versus comparator arm.

### Secondary Outcome Measures are:

1. PFS: increase in median PFS in experimental arm versus comparator arm.
2. Overall Response Rate (ORR) according to RECIST.
3. Disease Control Rate (DCR) for at least 12 weeks.
4. Time to First Subsequent Therapy (TFST): time from randomization to first subsequent therapy or death.
5. Progression-Free Survival 2 (PFS2): time from randomization to second objective disease progression or death.
6. Time to Second Subsequent Therapy (TSST): time from randomization to second subsequent therapy or death.
7. Patient Reported Outcomes (PROs) like Quality of Life questionnaire EORTC-QLQ-C30 & EORTC-QLQ-EN24. These are the validated questionnaires to be answered by patients. Results are reported as descriptive and on a scale of 1-10.
8. Number of participants with treatment-related adverse events as assessed by CTCAE v4.0.
9. Compliance in the two treatment arms.
10. Dose reductions/interruptions in the two treatment arms.

Toxicity/efficacy of the various compounds of the disclosure are analyzed and compared.

### Example 4: Dosing Regimen to Evaluate an Oral Pharmaceutical Composition Disclosed Herein with a Progestin in a Subject

The study is a randomized, double-blind, placebo-controlled, study of 8-cyclopentyl-2-((4-(4-methylpiperazin-1-yl)phenyl)amino)-7-oxo-7,8-dihydropyrido[2,3-d]pyrimidine-6-carbonitrile (Compound 1) in combination with a progestin (e.g., megestrol acetate) versus placebo in combination with a progestin (e.g., megestrol acetate) for patients with estrogen receptor positive advanced or recurrent ovarian cancer.

Study Design: This study is a treatment response study using 1:1 randomized double-blind study. Patients with ovarian cancer are randomized into one of two treatment arms: Arm A: (placebo): megestrol acetate-placebo combination therapy; and Arm B (experimental): megestrol acetate-Compound 1 combination therapy. Each cycle in the study is 28 days of treatment with tumors assessed every 12 weeks.

Compound 1 is given in the form of a hard capsule comprising 48.4 mg 8-cyclopentyl-2-((4-(4-methylpiperazin-1-yl)phenyl)amino)-7-oxo-7,8-dihydropyrido[2,3-d]pyrimidine-6-carbonitrilemonolactate salt, equivalent to 40 mg of 8-cyclopentyl-2-((4-(4-methylpiperazin-1-yl)phenyl)amino)-7-oxo-7,8-dihydropyrido[2,3-d]pyrimidine-6-carbonitrile. Compound 1 is taken once daily for 28 days (one cycle). Alternatively, Compound 1 is taken once daily continuously for 3 weeks with one week of no administration, for a total of 28 days. Treatment continues until progression of disease or unacceptable toxicity. Compound 1 is taken in the morning, on an empty stomach.

Megestrol acetate is given in the form of 625 mg of an oral suspension per day (5 mL of a 125 mg/mL suspension per day or one teaspoon daily). One 625 mg megestrol acetate dose is taken once on days 1-28 (one cycle). Treatment continues until progression of disease or unacceptable toxicity. Megestrol acetate is taken in the morning, at the same time as either Compound 1 or the placebo.

Primary outcome measures Primary outcome measures are increase in Progression-Free Survival (PFS) in experimental arm versus comparator arm.

Secondary Outcome Measures are:
1. PFS: increase in median PFS in experimental arm versus comparator arm.
2. Overall Response Rate (ORR) according to RECIST.
3. Disease Control Rate (DCR) for at least 12 weeks.
4. Time to First Subsequent Therapy (TFST): time from randomization to first subsequent therapy or death.
5. Progression-Free Survival 2 (PFS2): time from randomization to second objective disease progression or death.
6. Time to Second Subsequent Therapy (TSST): time from randomization to second subsequent therapy or death.
7. Patient Reported Outcomes (PROs) like Quality of Life questionnaire EORTC-QLQ-C30 & EORTC-QLQ-EN24. These outcomes are the validated questionnaires to be answered by patients. Results are reported as descriptive and on a scale of 1-10.
8. Number of participants with treatment-related adverse events as assessed by CTCAE v4.0.
9. Compliance in the two treatment arms.
10. Dose reductions/interruptions in the two treatment arms.

Toxicity/efficacy of the various compounds of the disclosure are analyzed and compared.

### Example 5 In Vitro Multi-Kinase Activity-IC₅₀ Values

The study evaluates the IC₅₀ values (in nM) of Compound 1 in comparison to FDA approved CDK 4/6 inhibitors Comparator-1, Comparator-2 and Comparator-3, which represent treatment advances for HR+ HER 2-metastatic breast cancer. IC₅₀ value is a quantitative measure indicating the concentration needed to inhibit the listed kinase by 50%. The results are shown in TABLE 1.

### Table 1: IC₅₀ Values (in nM)

TABLE 1 shows that Compound 1 had comparable IC₅₀ values for CDK4 and CDK6 as Comparator-1, Comparator-2, and Comparator-3. Compound 1 had significantly lower IC₅₀ values (1 to 3 magnitude lower) for CSFIR, ARK 5, and KIT, than Comparator-1, Comparator-2,

| | | | |
|---|---|---|---|
| Com- | Com- | Com- | Com- |
| pound 1 | parator-1 | parator-2 | parator-3 |

CDK Family

| | | | | |
|---|---|---|---|---|
| CDK4/cyclin D1 | 2 | 2 | 3 | 0.8 |
| CDK6/cyclin D1 | 0.6 | 0.8 | 2.0 | 0.6 |
| CDK1/cyclin B | 515 | >10,000 | >10,000 | 129 |
| CDK2/cyclin E | 69 | 2300 | >10,000 | 130 |
| CDK7/cyclin H | 3766 | >10,000 | >10,000 | 992 |
| CDK9/cyclinT1 | 48 | 630 | 390 | 7 |
| CDK16/cyclin Y | 25 | 2708 | >10,000 | 42 |
| CDK17/cyclin Y | 14 | 2528 | >10,000 | 29 |

Other Kinases

| | | | | |
|---|---|---|---|---|
| CSF1R | 0.7 | >10,000 | >10,000 | >10,000 |
| ARK 5/NUAK 1 | 5 | 1,400 | 1,540 | 773 |
| KIT | 8.5 | >10,000 | >10,000 | 7368 |

and Comparator-3. Compound 1 had also significantly lower IC₅₀ values (1 to 3 magnitude lower) for CDK 1, CDK 2, CDK 7, CDK 9, CDK 16, and CDK 17, than Comparator-1 and Comparator-2. This study demonstrates that Compound 1 has a high efficacy in inhibit a number of kinases.

### Example 6 Evaluate an Oral Pharmaceutical Composition Disclosed Herein with an Antiestrogen in a Subject with Low Grade Serous Ovarian Cancer

The study is a randomized, double-blind, placebo-controlled, study of 8-cyclopentyl-2-((4-(4-methylpiperazin-1-yl)phenyl)amino)-7-oxo-7,8-dihydropyrido[2,3-d]pyrimidine-6-carbonitrile (Compound 1) in combination with an antiestrogen (e.g., aromatase inhibitor (AI), selective estrogen receptor modulator (SERM), and selective estrogen receptor degrader (SERD) versus placebo in combination with an antiestrogen (e.g., AI, SERM, SERD) for patients with low grade serous ovarian cancer (LGSOC).

Study Design: This study is a treatment response study using 1:1 randomized double-blind study. Patients with LGSOC are randomized into one of two treatment arms: Arm A: (placebo): antiestrogen-placebo combination therapy; and Arm B (experimental): antiestrogen-Compound 1 combination therapy. Each cycle in the study is 28 days of treatment with tumors assessed every 12 weeks.

Compound 1 is given in the form of a hard capsule comprising 8-cyclopentyl-2-((4-(4-methylpiperazin-1-yl)phenyl)amino)-7-oxo-7,8-dihydropyrido[2,3-d]pyrimidine-6-carbonitrilemonolactate salt, equivalent to 40 mg of 8-cyclopentyl-2-((4-(4-methylpiperazin-1-yl)phenyl)amino)-7-oxo-7,8-dihydropyrido[2,3-d]pyrimidine-6-carbonitrile. Compound 1 is taken once daily for 28 days (one cycle). Alternatively, Compound 1 is taken once daily continuously for 3 weeks with one week of no administration, for a total of 28 days. Treatment continues until progression of disease or unacceptable toxicity. Compound 1 is taken in the morning, on an empty stomach.

Primary outcome measures Primary outcome measures are increase in Progression-Free Survival (PFS) in experimental arm versus comparator arm.

Secondary Outcome Measures are:
1. PFS: increase in median PFS in experimental arm versus comparator arm.
2. Overall Response Rate (ORR) according to RECIST.
3. Disease Control Rate (DCR) for at least 12 weeks.
4. Time to First Subsequent Therapy (TFST): time from randomization to first subsequent therapy or death.
5. Progression-Free Survival 2 (PFS2): time from randomization to second objective disease progression or death.
6. Time to Second Subsequent Therapy (TSST): time from randomization to second subsequent therapy or death.
7. Number of participants with treatment-related adverse events as assessed by CTCAE v4.0.
8. Compliance in the two treatment arms.

Toxicity/efficacy of the various compounds of the disclosure are analyzed and compared.

### Embodiments

Embodiment 1. A method of treating low grade serous ovarian cancer in a subject in need thereof, the method comprising administering to the subject a therapeutically-effective amount of a compound of formula (I)

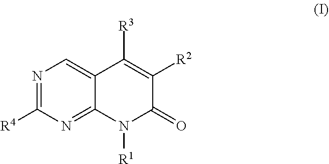

wherein:
   R¹ is alkyl, alkenyl, alkynyl, cycloalkyl, aryl, heteroaryl or heterocyclyl, each of which is unsubstituted or substituted, or hydrogen;
   R² is alkyl, alkenyl, alkynyl, cycloalkyl, aryl, heteroaryl, heterocyclyl, —CN, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen;
   R³ is alkyl, alkenyl, alkynyl, cycloalkyl, aryl, heterocyclyl or heteroaryl, each of which is unsubstituted or substituted, or hydrogen or halogen;
   R⁴ is alkyl, alkenyl, alkynyl, cycloalkyl, aryl, heterocyclyl, heteroaryl, —CN, C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
   each R⁵ and R⁶ is independently alkyl, alkenyl, alkynyl, cycloalkyl, aryl, heterocyclyl, or heteroaryl, each of which is independently substituted or unsubstituted, or hydrogen or halogen;
provided that, when R⁴ is —NR⁵R⁶ and one of R⁵ and R⁶ is pyridyl, then R² is not —C(O)CH₃,
or a pharmaceutically-acceptable salt thereof.

Embodiment 2. The method of embodiment 1, wherein R¹ is cycloalkyl.

Embodiment 3. The method of embodiment 1 or 2, wherein R¹ is cyclopentyl.

Embodiment 4. The method of any one of embodiments 1-3, wherein R² is CN.

Embodiment 5. The method of any one of embodiments 1-4, wherein R³ is hydrogen.

Embodiment 6. The method of any one of embodiments 1-5, wherein R⁴ is —NR⁵R⁶.

Embodiment 7. The method of any one of embodiments 1-6, wherein one of R⁵ and R⁶ is hydrogen.

Embodiment 8. The method of any one of embodiments 1-7, wherein one of R³ and R⁶ is phenyl.

Embodiment 9. The method of any one of embodiments 1-8, wherein one of R⁵ and R⁶ is phenyl substituted with heterocyclyl.

Embodiment 10, any one of embodiments 1-9, wherein one of R⁵ and R⁶ is phenyl substituted with piperazinyl.

Embodiment 11, any one of embodiments 1-10, wherein one of R⁵ and R⁶ is phenyl substituted with 4-methyl piperazinyl.

Embodiment 12. The method of embodiment 1, wherein R⁴ is

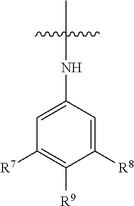

wherein:
   R⁷ is alkyl, alkenyl, alkynyl, cycloalkyl, aryl, heterocyclyl, heteroaryl, —OR⁵, —SR⁵, or —NR⁵R⁶, each of which is unsubstituted or substituted, or hydrogen;
   R⁸ is alkyl, alkenyl, alkynyl, cycloalkyl, aryl, heterocyclyl, heteroaryl, —OR⁵, —SR⁵, or —NR⁵R⁶, each of which is unsubstituted or substituted, or hydrogen; and
   R⁹ is alkyl, alkenyl, alkynyl, cycloalkyl, aryl, heterocyclyl, heteroaryl, —OR⁵, —SR⁵, or —NR⁵R⁶, each of which is unsubstituted or substituted, or hydrogen.

Embodiment 13. The method of embodiment 12, wherein R⁷ is hydrogen.

Embodiment 14. The method of embodiment 12 or 13, wherein R⁸ is hydrogen.

Embodiment 15. The method of any one of embodiments 12-14, wherein R⁹ unsubstituted or substituted heterocyclyl.

Embodiment 16. The method of any one of embodiments 12-15, wherein R⁹ is unsubstituted or substituted piperazinyl.

Embodiment 17. The method of any one of embodiments 12-16, wherein R⁹ is 4-methyl piperazinyl.

Embodiment 18. The method of any one of embodiments 12-17, wherein the compound is a compound of formula (II)

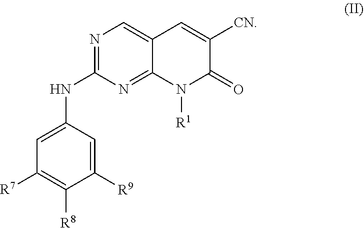

Embodiment 19. The method of any one of embodiments 12-18, wherein the compound is a compound of formula (III)

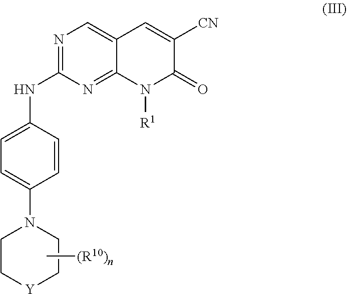

wherein:
Y is O, S, or NR¹¹;
each R¹⁰ is independently alkyl, alkenyl, alkynyl, cycloalkyl, aryl, heterocyclyl, heteroaryl, —OR⁵, —SR⁵, or —NR⁵R⁶, each of which is unsubstituted or substituted; R¹¹ is alkyl, alkenyl, alkynyl, cycloalkyl, aryl, heterocyclyl, or heteroaryl, each of
which is independently substituted or unsubstituted, or hydrogen or halogen; and
n is 0, 1, 2, 3, 4, 5, 6, 7, or 8.

Embodiment 20. The method of embodiment 19, wherein R¹ is cycloalkyl.

Embodiment 21. The method of embodiment 19 or 20, wherein R¹ is cyclopentyl.

Embodiment 22. The method of embodiment 19-21, wherein Y is NR¹¹.

Embodiment 23. The method of embodiment 22, wherein R¹¹ is alkyl.

Embodiment 24. The method of embodiment 22 or 23, wherein R¹¹ is methyl.

Embodiment 25. The method of any one of embodiments 19-24, wherein n is 0.

Embodiment 26. The method of any one of embodiments 1-25, wherein the compound is 8-cyclopentyl-2-((4-(4-methylpiperazin-1-yl)phenyl)amino)-7-oxo-7,8-dihydropyrido[2,3-d]pyrimidine-6-carbonitrile, or a pharmaceutically-acceptable salt thereof.

Embodiment 27. The method any one of embodiments 1-26, wherein the compound is 8-cyclopentyl-2-((4-(4-methylpiperazin-1-yl)phenyl)amino)-7-oxo-7,8-dihydropyrido[2,3-d]pyrimidine-6-carbonitrile monolactate.

Embodiment 28. The method of any one of embodiments 1-27, wherein the therapeutically-effective amount is about 80 mg to about 500 mg per day.

Embodiment 29. The method of any one of embodiments 1-28, wherein the low grade serous ovarian carcinoma is an ovarian carcinoma.

Embodiment 30. The method of any one of embodiments 1-28, wherein the low grade serous ovarian carcinoma is a fallopian tube carcinoma.

Embodiment 31. The method of any one of embodiments 1-28, wherein the low grade serous ovarian carcinoma is a primary peritoneal carcinoma.

Embodiment 32. The method of any one of embodiments 1-30, wherein the low grade serous ovarian carcinoma is a hormone receptor positive low grade serous ovarian carcinoma.

Embodiment 33. The method of any one of embodiments 1-31, wherein the low grade serous ovarian carcinoma is an estrogen receptor positive low grade serous ovarian carcinoma.

Embodiment 34. The method of any one of embodiments 1-31, wherein the low grade serous ovarian carcinoma is a progesterone receptor positive low grade serous ovarian carcinoma.

Embodiment 35. The method of any one of embodiments 1-34, wherein the subject received a therapy other than the compound for the low grade serous ovarian carcinoma prior to the administering.

Embodiment 36. The method of embodiment 35, wherein the therapy was received after the subject was diagnosed with the low grade serous ovarian carcinoma.

Embodiment 37. The method of embodiment 35 or 36, wherein the subject has not responded to the therapy.

Embodiment 38. The method of embodiment 35 or 36, wherein the subject experienced a relapse of the low grade serous ovarian carcinoma after the therapy.

Embodiment 39. The method of any one of embodiments 1-38, wherein the administering is oral.

Embodiment 40. The method of any one of embodiments 1-38, wherein the administering is intravenous.

Embodiment 41. The method of any one of embodiments 1-40, wherein the administering is once daily.

Embodiment 42. The method of any one of embodiments 1-41, wherein the administering is once daily for at least 4 weeks.

Embodiment 43. The method of any one of embodiments 1-41, wherein the administering is oral on a 4-week cycle of: (i) a continuous, three-week period of once-daily administration; and ii) immediately following the three-week period, one week of no administration.

Embodiment 44. The method of any one of embodiments 1-41, wherein the administering is oral on a 4-week cycle of: (i) a continuous, three-week period of once-daily, morning administration, wherein the therapeutically-effective amount is from about 40 mg to about 500 mg; and ii) immediately following the three-week period, one week of no administration.

Embodiment 45. The method of any one of embodiments 1-44, wherein the compound is administered in a pharmaceutical composition, wherein the pharmaceutical composition is in a unit dosage form, the unit dosage form further comprising a pharmaceutically-acceptable excipient.

Embodiment 46. The method of embodiment 45, wherein the unit dosage form comprises about 40 mg of 8-cyclopentyl-2-((4-(4-methylpiperazin-1-yl)phenyl)amino)-7-oxo-7,8-dihydropyrido[2,3-d]pyrimidine-6-carbonitrile.

Embodiment 47. The method of embodiment 45 or 46, wherein unit dosage form comprises about 48.4 mg of 8-cyclopentyl-2-((4-(4-methylpiperazin-1-yl)phenyl)amino)-7-oxo-7,8-dihydropyrido[2,3-d]pyrimidine-6-carbonitrile monolactate.

Embodiment 48. The method of any one of embodiments 45-47, wherein the unit dosage form is a capsule.

Embodiment 49. The method of any one of embodiments 1-48, wherein the administering occurs in a morning of a day.

Embodiment 50. The method of any one of embodiments 1-49, wherein the subject is in a fasted state.

Embodiment 51. The method of any one of embodiments 1-50, further comprising administering to the subject a therapeutically-effective amount of a second compound.

Embodiment 52. The method of embodiment 51, wherein the second compound is an estrogen receptor modulator.

Embodiment 53. The method of embodiment 51 or 52, wherein the second compound is an estrogen receptor blocker.

Embodiment 54. The method of any one of embodiments 51-53, wherein the second compound is an aromatase inhibitor.

Embodiment 55. The method of any one of embodiments 51-54, wherein the second compound is letrozole or a pharmaceutically-acceptable salt thereof.

Embodiment 56. The method of any one of embodiments 51-55, wherein the second compound is administered in a pharmaceutical composition, wherein the pharmaceutical composition is in a unit dosage form, the unit dosage form further comprising a pharmaceutically-acceptable excipient.

Embodiment 57. The method of embodiment 56, wherein the unit dosage form comprises about 2.5 mg of letrozole.

Embodiment 58. The method of embodiment 56 or 57, the unit dosage form is a tablet.

Embodiment 59. The method of embodiment 51, wherein the second compound is a progestin.

Embodiment 60. The method of embodiment 59, wherein the progestin is megestrol acetate.

Embodiment 61. The method of embodiment 59 or 60, wherein megestrol acetate is administered in a pharmaceutical composition, wherein the pharmaceutical composition is in a unit dosage form, the unit dosage form further comprising a pharmaceutically-acceptable excipient.

Embodiment 62. The method of embodiment 61, wherein the unit dosage form comprises about 125 mg/mL of megestrol acetate.

Embodiment 63. The method of embodiment 61 or 62, wherein the unit dosage form is an oral suspension.

Embodiment 64. The method of any one of embodiments 61-63, wherein the megestrol acetate is administered at a dose of about 625 mg per day.

Embodiment 65. The method of embodiment 51, wherein the second compound is an Estrogen Receptor Degrader.

Embodiment 66. The method of embodiment 65, wherein the second compound is selected from tamoxifen, toremifene and raloxifene.

Embodiment 67. The method of any one of embodiments 51-66, wherein the administering of the second compound is once daily.

Embodiment 68. The method of embodiment 51, wherein the second compound is a selective estrogen receptor degrader.

Embodiment 69. The method of embodiment 51, wherein the second compound is a vascular endothelial growth factor inhibitor.

Embodiment 70. The method of embodiment 51, wherein the second compound is a phosphoinositide 3-kinase inhibitor.

Embodiment 71. The method of any one of embodiments embodiment 51-70, wherein the administering of the second compound is oral.

Embodiment 72. The method of any one of embodiments embodiment 51-70, wherein the administering of the second compound is intravenous.

Embodiment 73. The method of any one of embodiments embodiment 51-70, wherein the administering of the compound of formula (I) is oral, and the administering of the second compound is oral.

Embodiment 74. The method of any one of embodiments embodiment 51-73, wherein the administering of the compound of formula (I) is prior to the administering of the second compound.

Embodiment 75. The method of any one of embodiments embodiment 51-73, wherein the administering of the second compound is prior to the administering of the compound of formula (I).

Embodiment 76. The method of any one of embodiments embodiment 51-73, wherein the administering of the compound of formula (I) is concurrently with the administering of the second compound.

Embodiment 77. A method of treating high grade serous ovarian cancer in a subject in need thereof, the method comprising administering to the subject a therapeutically-effective amount of a compound of formula (I)

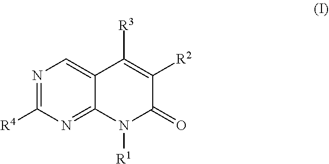

wherein:
   R¹ is alkyl, alkenyl, alkynyl, cycloalkyl, aryl, heteroaryl or heterocyclyl, each of which is unsubstituted or substituted, or hydrogen;
   R² is alkyl, alkenyl, alkynyl, cycloalkyl, aryl, heteroaryl, heterocyclyl, —CN, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen;
   R³ is alkyl, alkenyl, alkynyl, cycloalkyl, aryl, heterocyclyl or heteroaryl, each of which is unsubstituted or substituted, or hydrogen or halogen;
   R⁴ is alkyl, alkenyl, alkynyl, cycloalkyl, aryl, heterocyclyl, heteroaryl, —CN, C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
   each R⁵ and R⁶ is independently alkyl, alkenyl, alkynyl, cycloalkyl, aryl, heterocyclyl, or heteroaryl, each of which is independently substituted or unsubstituted, or hydrogen or halogen;
provided that, when R⁴ is —NR⁵R⁶ and one of R⁵ and R⁶ is pyridyl, then R² is not —C(O)CH₃,
or a pharmaceutically-acceptable salt thereof.

Embodiment 78. The method of embodiment 77, wherein R¹ is cycloalkyl.

Embodiment 79. The method of embodiment 77 or 78, wherein R¹ is cyclopentyl.

Embodiment 80. The method of any one embodiments 77-79, wherein R² is CN.

Embodiment 81. The method of any one embodiments 77-80, wherein R³ is hydrogen.

Embodiment 82. The method of any one embodiments 77-81, wherein R⁴ is —NR⁵R⁶.

Embodiment 83. The method of any one embodiments 77-82, wherein one of R⁵ and R⁶ is hydrogen.

Embodiment 84. The method of any one embodiments 77-83, wherein one of R⁵ and R⁶ is phenyl.

Embodiment 85. The method of any one embodiments 77-84, wherein one of R⁵ and R⁶ is phenyl substituted with heterocyclyl.

Embodiment 86. The method of any one embodiments 77-85, wherein one of R⁵ and R⁶ is phenyl substituted with piperazinyl.

Embodiment 87. The method of any one embodiments 77-86, wherein one of R⁵ and R⁶ is phenyl substituted with 4-methyl piperazinyl.

Embodiment 88. The method of embodiment 77, wherein R⁴ is

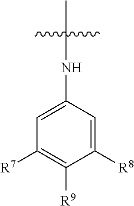

wherein:
   R⁷ is alkyl, alkenyl, alkynyl, cycloalkyl, aryl, heterocyclyl, heteroaryl, —OR⁵, —SR⁵, or —NR⁵R⁶, each of which is unsubstituted or substituted, or hydrogen;
   R⁸ is alkyl, alkenyl, alkynyl, cycloalkyl, aryl, heterocyclyl, heteroaryl, —OR⁵, —SR⁵, or —NR⁵R⁶, each of which is unsubstituted or substituted, or hydrogen; and
   R⁹ is alkyl, alkenyl, alkynyl, cycloalkyl, aryl, heterocyclyl, heteroaryl, —OR⁵, —SR⁵, or —NR⁵R⁶, each of which is unsubstituted or substituted, or hydrogen.

Embodiment 89. The method of embodiment 88, wherein R⁷ is hydrogen.

Embodiment 90. The method of embodiment 88 or 89, wherein R⁸ is hydrogen.

Embodiment 91. The method of any one of embodiments 88-90, wherein R⁹ unsubstituted or substituted heterocyclyl.

Embodiment 92. The method of any one of embodiments 88-91, wherein R⁹Is unsubstituted or substituted piperazinyl.

Embodiment 93. The method of any one of embodiments 88-92, wherein R⁹ is 4-methyl piperazinyl.

Embodiment 94. The method of any one of embodiments 88-93, wherein the compound is a compound of formula (II)

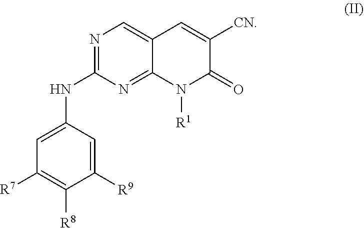

Embodiment 95. The method of The method of any one of embodiments 88-94, wherein the compound is a compound of formula (III)

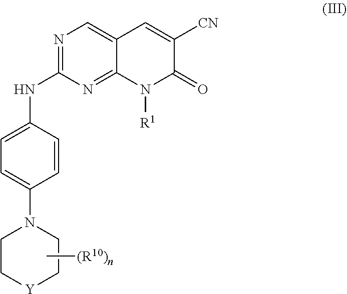

wherein:
Y is O, S, or NR¹¹;
each R¹⁰ is independently alkyl, alkenyl, alkynyl, cycloalkyl, aryl, heterocyclyl, heteroaryl, —OR⁵, —SR⁵, or —NR⁵R⁶, each of which is unsubstituted or substituted;
R¹¹ is alkyl, alkenyl, alkynyl, cycloalkyl, aryl, heterocyclyl, or heteroaryl, each of which is independently substituted or unsubstituted, or hydrogen or halogen; and
n is 0, 1, 2, 3, 4, 5, 6, 7, or 8.

Embodiment 96. The method of embodiment 95, wherein R¹ is cycloalkyl.

Embodiment 97. The method of embodiment 95 or 96, wherein R¹ is cyclopentyl.

Embodiment 98. The method of any one embodiments 95-97, wherein Y is NR¹¹.

Embodiment 99. The method of embodiment 98, wherein R¹¹ is alkyl.

Embodiment 100. The method of embodiment 98 or 99, wherein R¹¹ is methyl.

Embodiment 101. The method of any one of embodiments 95-100, wherein n is 0.

Embodiment 102. The method of any one of embodiments 77-101, wherein the compound is 8-cyclopentyl-2-((4-(4-methylpiperazin-1-yl)phenyl)amino)-7-oxo-7,8-dihydropyrido[2,3-d]pyrimidine-6-carbonitrile, or a pharmaceutically-acceptable salt thereof.

Embodiment 103. The method of any one of embodiments 75-100, wherein the compound is 8-cyclopentyl-2-((4-(4-methylpiperazin-1-yl)phenyl)amino)-7-oxo-7,8-dihydropyrido[2,3-d]pyrimidine-6-carbonitrile monolactate.

Embodiment 104. The method of any one of embodiments 77-103, wherein the therapeutically-effective amount is about 80 mg to about 500 mg per day.

Embodiment 105. The method of any one of embodiments 77-104, wherein the high grade serous ovarian carcinoma is a hormone receptor positive high grade serous ovarian carcinoma.

Embodiment 106. The method of any one of embodiments 77-105, wherein the high grade serous ovarian carcinoma is an estrogen receptor positive high grade serous ovarian carcinoma.

Embodiment 107. The method of any one of embodiments 77-105, the high grade serous ovarian carcinoma is a progesterone receptor positive high grade serous ovarian carcinoma.

Embodiment 108. The method of any one of embodiments 77-107, wherein the subject received a therapy other than the compound for the high grade serous ovarian carcinoma prior to the administering.

Embodiment 109. The method of embodiment 108, wherein the therapy was received after the subject was diagnosed with the high grade serous ovarian carcinoma.

Embodiment 110. The method of embodiment 108, wherein the subject has not responded to the therapy.

Embodiment 111. The method of embodiment 108, wherein the subject experienced a relapse of the high grade serous ovarian carcinoma after the therapy.

Embodiment 112. The method of any one of embodiments 77-111, wherein the administering is oral.

Embodiment 113. The method of any one of embodiments 77-111, wherein the administering is intravenous.

Embodiment 114. The method of any one of embodiments 77-113, wherein the administering is once daily.

Embodiment 115. The method of any one of embodiments 77-114, wherein the administering is once daily for at least 4 weeks.

Embodiment 116. The method of any one of embodiments 77-114, wherein the administering is oral on a 4-week cycle of: (i) a continuous, three-week period of once-daily administration; and ii) immediately following the three-week period, one week of no administration.

Embodiment 117. The method of any one of embodiments 77-114, wherein the administering is oral on a 4-week cycle of: (i) a continuous, three-week period of once-daily, morning administration, wherein the therapeutically-effective amount is from about 80 mg to about 500 mg; and ii) immediately following the three-week period, one week of no administration.

Embodiment 118. The method of any one of embodiments 77-117, wherein the compound is administered in a pharmaceutical composition, wherein the pharmaceutical composition is in a unit dosage form, the unit dosage form further comprising a pharmaceutically-acceptable excipient.

Embodiment 119. The method of embodiment 118, wherein the unit dosage form comprises about 40 mg of 8-cyclopentyl-2-((4-(4-methylpiperazin-1-yl)phenyl)amino)-7-oxo-7,8-dihydropyrido[2,3-d]pyrimidine-6-carbonitrile.

Embodiment 120. The method of embodiment 118 or 119, wherein unit dosage form comprises about 48.4 mg of 8-cyclopentyl-2-((4-(4-methylpiperazin-1-yl)phenyl)amino)-7-oxo-7,8-dihydropyrido[2,3-d]pyrimidine-6-carbonitrile monolactate.

Embodiment 121. The method of any one of embodiments 118-120, wherein the unit dosage form is a capsule.

Embodiment 122. The method of any one of embodiments 77-121, wherein the administering occurs in a morning of a day.

Embodiment 123. The method of embodiment any one of embodiments 77-122, wherein the subject is in a fasted state.

Embodiment 124. The method of any one of embodiments 77-123, further comprising administering to the subject a therapeutically-effective amount of a second compound.

Embodiment 125. The method of embodiment 124, wherein the second compound is an estrogen receptor modulator.

Embodiment 126. The method of embodiment 124 or 125, wherein the second compound is an estrogen receptor blocker.

Embodiment 127. The method of any one of embodiments 124-126, wherein the second compound is an aromatase inhibitor.

Embodiment 128. The method of any one of embodiments 124-127, wherein the second compound is letrozole or a pharmaceutically-acceptable salt thereof.

Embodiment 129. The method of any one of embodiments 124-128, wherein the second compound is administered in a pharmaceutical composition, wherein the pharmaceutical composition is in a unit dosage form, the unit dosage form further comprising a pharmaceutically-acceptable excipient.

Embodiment 130. The method of embodiment 129, wherein the unit dosage form comprises about 2.5 mg of letrozole.

Embodiment 131. The method of embodiment 129 or 130, the unit dosage form is a tablet.

Embodiment 132. The method of embodiment 124, wherein the second compound is a progestin.

Embodiment 133. The method of embodiment 132, wherein the progestin is megestrol acetate.

Embodiment 134. The method of embodiment 133, wherein megestrol acetate is administered in a pharmaceutical composition, wherein the pharmaceutical composition is in a unit dosage form, the unit dosage form further comprising a pharmaceutically-acceptable excipient.

Embodiment 135. The method of embodiment 134, wherein the unit dosage form comprises about 125 mg/mL of megestrol acetate.

Embodiment 136. The method of embodiment 134 or 135, wherein the unit dosage form is an oral suspension.

Embodiment 137. The method of any one of embodiments 134-136, wherein the megestrol acetate is administered at a dose of about 625 mg per day.

Embodiment 138. The method of any one of embodiments 124-137, wherein the administering of the second compound is once daily.

Embodiment 139. The method of embodiment 124, wherein the second compound is a selective estrogen receptor degrader.

Embodiment 140. The method of embodiment 124, wherein the second compound is a vascular endothelial growth factor inhibitor.

Embodiment 141. The method of embodiment 124, wherein the second compound is a phosphoinositide 3-kinase inhibitor.

Embodiment 142. The method of any one of embodiments 124-141, wherein the administering of the second compound is oral.

Embodiment 143. The method of any one of embodiments 124-141, wherein the administering of the second compound is intravenous.

Embodiment 144. The method of any one of embodiments 124-141, wherein the administering of the compound of formula (I) is oral, and the administering of the second compound is oral.

Embodiment 145. The method of any one of embodiments 124-144, wherein the administering of the compound of formula (I) is prior to the administering of the second compound.

Embodiment 146. The method of any one of embodiments 124-144, wherein the administering of the second compound is prior to the administering of the compound of formula (I).

Embodiment 147. The method of any one of embodiments 124-144, wherein the administering of the compound of formula (I) is concurrently with the administering of the second compound.

Embodiment 148. A method of treating ovarian cancer in a subject in need thereof, the method comprising administering to the subject a therapeutically-effective amount of a compound of formula (I)

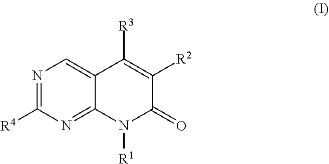

wherein:
   R¹ is alkyl, alkenyl, alkynyl, cycloalkyl, aryl, heteroaryl or heterocyclyl, each of which is unsubstituted or substituted, or hydrogen;
   R² is alkyl, alkenyl, alkynyl, cycloalkyl, aryl, heteroaryl, heterocyclyl, —CN, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen;
   R³ is alkyl, alkenyl, alkynyl, cycloalkyl, aryl, heterocyclyl or heteroaryl, each of which is unsubstituted or substituted, or hydrogen or halogen;
   R⁴ is alkyl, alkenyl, alkynyl, cycloalkyl, aryl, heterocyclyl, heteroaryl, —CN, C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
   each R⁵ and R⁶ is independently alkyl, alkenyl, alkynyl, cycloalkyl, aryl, heterocyclyl, or heteroaryl, each of which is independently substituted or unsubstituted, or hydrogen or halogen;
provided that, when R⁴ is —NR⁵R⁶ and one of R⁵ and R⁶ is pyridyl, then R² is not —C(O)CH₃,
   or a pharmaceutically-acceptable salt thereof;
wherein the subject received a therapy other than the compound for the ovarian cancer prior to the administering.

Embodiment 149. The method of embodiment 148, wherein R¹ is cycloalkyl.

Embodiment 150. The method of embodiment 148 or 149, wherein R¹ is cyclopentyl.

Embodiment 151. The method of any one of embodiments 148-150, wherein R² is CN.

Embodiment 152. The method of any one of embodiments 148-151, wherein R³ is hydrogen.

Embodiment 153. The method of any one of embodiments 148-152, wherein R⁴ is —NR⁵R⁶.

Embodiment 154. The method of embodiment 153, wherein one of R⁵ and R⁶ is hydrogen.

Embodiment 155. The method of embodiment 153 or 154, wherein one of R⁵ and R⁶ is phenyl.

Embodiment 156. The method of any one of embodiments 153-155, wherein one of R⁵ and R⁶ is phenyl substituted with heterocyclyl.

Embodiment 157. The method of any one of embodiments 153-156, wherein one of R⁵ and R⁶ is phenyl substituted with piperazinyl.

Embodiment 158. The method of any one of embodiments 153-157, wherein one of R⁵ and R⁶ is phenyl substituted with 4-methyl piperazinyl.

Embodiment 159. The method of embodiment 148, wherein R⁴ is

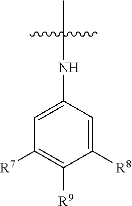

wherein:
   R⁷ is alkyl, alkenyl, alkynyl, cycloalkyl, aryl, heterocyclyl, heteroaryl, —OR⁵, —SR⁵, or —NR⁵R⁶, each of which is unsubstituted or substituted, or hydrogen;
   R⁸ is alkyl, alkenyl, alkynyl, cycloalkyl, aryl, heterocyclyl, heteroaryl, —OR⁵, —SR⁵, or —NR⁵R⁶, each of which is unsubstituted or substituted, or hydrogen; and
   R⁹ is alkyl, alkenyl, alkynyl, cycloalkyl, aryl, heterocyclyl, heteroaryl, —OR⁵, —SR⁵, or —NR⁵R⁶, each of which is unsubstituted or substituted, or hydrogen.

Embodiment 160. The method of embodiment 159, wherein R⁷ is hydrogen.

Embodiment 161. The method of embodiment 159 or 160, wherein R⁸ is hydrogen.

Embodiment 162. The method of any one of embodiments 159-161, wherein R⁹ unsubstituted or substituted heterocyclyl.

Embodiment 163. The method of any one of embodiments 159-162, wherein R⁹ is unsubstituted or substituted piperazinyl.

Embodiment 164. The method of any one of embodiments 159-163, wherein R⁹ is 4-methyl piperazinyl.

Embodiment 165. The method of any one of embodiments 159-164, wherein the compound is a compound of formula (II)

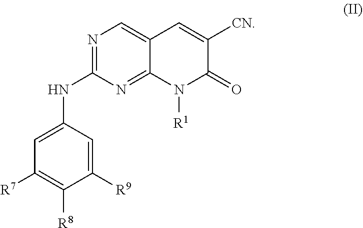

Embodiment 166. The method of embodiment 159, wherein the compound is a compound of formula (III)

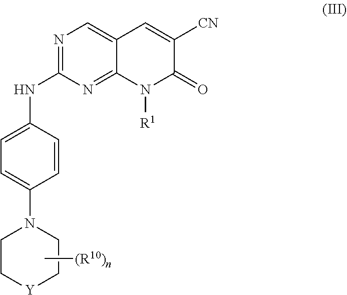

wherein:
Y is O, S, or NR¹¹;
each R¹⁰ is independently alkyl, alkenyl, alkynyl, cycloalkyl, aryl, heterocyclyl, heteroaryl, —OR⁵, —SR⁵, or —NR⁵R⁶, each of which is unsubstituted or substituted;
R¹¹ is alkyl, alkenyl, alkynyl, cycloalkyl, aryl, heterocyclyl, or heteroaryl, each of which is independently substituted or unsubstituted, or hydrogen or halogen; and
n is 0, 1, 2, 3, 4, 5, 6, 7, or 8.

Embodiment 167. The method of embodiment 166, wherein R¹ is cycloalkyl.

Embodiment 168. The method of embodiment 166 or 167, wherein R¹ is cyclopentyl.

Embodiment 169. The method of any one of embodiments 166-168, wherein Y is NR¹¹.

Embodiment 170. The method of embodiment 169, wherein R¹¹ is alkyl.

Embodiment 171. The method of embodiment 169 or 170, wherein R¹¹ is methyl.

Embodiment 172. The method of any one of embodiments 166-171, wherein n is 0.

Embodiment 173. The method of any one of embodiments 148-172, wherein the compound is 8-cyclopentyl-2-((4-(4-methylpiperazin-1-yl)phenyl)amino)-7-oxo-7,8-dihydropyrido[2,3-d]pyrimidine-6-carbonitrile, or a pharmaceutically-acceptable salt thereof.

Embodiment 174. The method of any one of embodiments 148-173, wherein the compound is 8-cyclopentyl-2-((4-(4-methylpiperazin-1-yl)phenyl)amino)-7-oxo-7,8-dihydropyrido[2,3-d]pyrimidine-6-carbonitrile monolactate.

Embodiment 175. The method of any one of embodiments 148-174, wherein the therapeutically-effective amount is about 80 mg to about 500 mg per day.

Embodiment 176. The method of any one of embodiments 148-175, wherein the ovarian cancer is a high grade serous ovarian carcinoma.

Embodiment 177. The method of any one of embodiments 148-175, wherein the ovarian cancer is a low grade serous ovarian carcinoma.

Embodiment 178. The method of embodiment 177, wherein the low grade serous ovarian carcinoma is an ovarian carcinoma.

Embodiment 179. The method of embodiment 177, wherein the low grade serous ovarian carcinoma is a fallopian tube carcinoma.

Embodiment 180. The method of embodiment 177, wherein the low grade serous ovarian carcinoma is a primary peritoneal carcinoma.

Embodiment 181. The method of any one of embodiments 148-179, wherein the ovarian cancer is a hormone receptor positive ovarian cancer.

Embodiment 182. The method of any one of embodiments 148-181, wherein the ovarian cancer is an estrogen receptor positive ovarian cancer.

Embodiment 183. The method of any one of embodiments 148-181, wherein the ovarian cancer is a progesterone receptor positive ovarian cancer.

Embodiment 184. The method of any one of embodiments 148-183, wherein the therapy was received after the subject was diagnosed with the ovarian cancer.

Embodiment 185. The method of any one of embodiments 148-183, wherein the subject has not responded to the therapy.

Embodiment 186. The method of any one of embodiments 148-183, wherein the subject experienced a relapse of the ovarian cancer after the therapy.

Embodiment 187. The method of any one of embodiments 148-186, wherein the administering is oral.

Embodiment 188. The method of any one of embodiments 148-186, wherein the administering is intravenous.

Embodiment 189. The method of any one of embodiments 148-188, wherein the administering is once daily.

Embodiment 190. The method of any one of embodiments 148-189, wherein the administering is once daily for at least 4 weeks.

Embodiment 191. The method of any one of embodiments 148-189, wherein the administering is oral on a 4-week cycle of: (i) a continuous, three-week period of once-daily administration; and ii) immediately following the three-week period, one week of no administration.

Embodiment 192. The method of any one of embodiments 148-189, wherein the administering is oral on a 4-week cycle of: (i) a continuous, three-week period of once-daily, morning administration, wherein the therapeutically-effective amount is from about 40 mg to about 500 mg; and ii) immediately following the three-week period, one week of no administration.

Embodiment 193. The method of any one of embodiments 148-192, wherein the compound is administered in a pharmaceutical composition, wherein the pharmaceutical composition is in a unit dosage form, the unit dosage form further comprising a pharmaceutically-acceptable excipient.

Embodiment 194. The method of embodiment 193, wherein the unit dosage form comprises about 40 mg of 8-cyclopentyl-2-((4-(4-methylpiperazin-1-yl)phenyl)amino)-7-oxo-7,8-dihydropyrido[2,3-d]pyrimidine-6-carbonitrile.

Embodiment 195. The method of embodiment 193 or 194, wherein unit dosage form comprises about 48.4 mg of 8-cyclopentyl-2-((4-(4-methylpiperazin-1-yl)phenyl)amino)-7-oxo-7,8-dihydropyrido[2,3-d]pyrimidine-6-carbonitrile monolactate.

Embodiment 196. The method of any one of embodiments 193-195, wherein the unit dosage form is a capsule.

Embodiment 197. The method of any one of embodiments 193-196, wherein the administering occurs in a morning of a day.

Embodiment 198. The method of any one of embodiments 193-197, wherein the subject is in a fasted state.

Embodiment 199. The method of any one of embodiments 193-198, further comprising administering to the subject a therapeutically-effective amount of a second compound.

Embodiment 200. The method of embodiment 199, wherein the second compound is an estrogen receptor modulator.

Embodiment 201. The method of embodiment 199 or 200, wherein the second compound is an estrogen receptor blocker.

Embodiment 202. The method of any one of embodiments 199-201, wherein the second compound is an aromatase inhibitor.

Embodiment 203. The method of any one of embodiments 199-202, wherein the second compound is letrozole or a pharmaceutically-acceptable salt thereof.

Embodiment 204. The method of any one of embodiments 199-203, wherein the second compound is administered in a pharmaceutical composition, wherein the pharmaceutical composition is in a unit dosage form, the unit dosage form further comprising a pharmaceutically-acceptable excipient.

Embodiment 205. The method of embodiment 204, wherein the unit dosage form comprises about 2.5 mg of letrozole.

Embodiment 206. The method of embodiment 204 or 205, the unit dosage form is a tablet.

Embodiment 207. The method of embodiment 199, wherein the second compound is a progestin.

Embodiment 208. The method of embodiment 207, wherein the progestin is megestrol acetate.

Embodiment 209. The method of embodiment 208, wherein megestrol acetate is administered in a pharmaceutical composition, wherein the pharmaceutical composition is in a unit dosage form, the unit dosage form further comprising a pharmaceutically-acceptable excipient.

Embodiment 210. The method of embodiment 208 or 209, wherein the unit dosage form comprises about 125 mg/mL of megestrol acetate.

Embodiment 211. The method of embodiment any one of embodiments 209 or 211, wherein the unit dosage form is an oral suspension.

Embodiment 212. The method of any one of embodiments 209-211, wherein the megestrol acetate is administered at a dose of about 625 mg per day.

Embodiment 213. The method of any one of embodiments 199-212, wherein the administering of the second compound is once daily.

Embodiment 214. The method of embodiment 199, wherein the second compound is a selective estrogen receptor degrader.

Embodiment 215. The method of embodiment 199, wherein the second compound is a vascular endothelial growth factor inhibitor.

Embodiment 216. The method of embodiment 199, wherein the second compound is a phosphoinositide 3-kinase inhibitor.

Embodiment 217. The method of any one of embodiments 199-216, wherein the administering of the second compound is oral.

Embodiment 218. The method of any one of embodiments 199-216, wherein the administering of the second compound is intravenous.

Embodiment 219. The method of any one of embodiments 199-216, wherein the administering of the compound of formula (I) is oral, and the administering of the second compound is oral.

Embodiment 220. The method of any one of embodiments 199-219, wherein the administering of the compound of formula (I) is prior to the administering of the second compound.

Embodiment 221. The method of any one of embodiments 199-219, wherein the administering of the second compound is prior to the administering of the compound of formula (I).

Embodiment 222. The method of any one of embodiments 199-219, wherein the administering of the compound of formula (I) is concurrently with the administering of the second compound.

Embodiment 223. A method of treating ovarian cancer in a subject in need thereof, the method comprising administering to the subject a therapeutically-effective amount of a compound of formula (I)

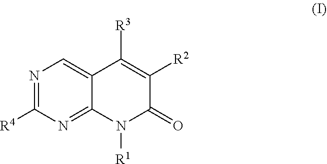

wherein:
   R¹ is alkyl, alkenyl, alkynyl, cycloalkyl, aryl, heteroaryl or heterocyclyl, each of which is unsubstituted or substituted, or hydrogen;
   R² is alkyl, alkenyl, alkynyl, cycloalkyl, aryl, heteroaryl, heterocyclyl, —CN, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen;
   R³ is alkyl, alkenyl, alkynyl, cycloalkyl, aryl, heterocyclyl or heteroaryl, each of which is unsubstituted or substituted, or hydrogen or halogen;
   R⁴ is alkyl, alkenyl, alkynyl, cycloalkyl, aryl, heterocyclyl, heteroaryl, —CN, C(O)R⁵, —C(O)OR⁵, —C(O)NR⁵R⁶, —C(═N)NR⁵R⁶, —OR⁵, —SR⁵, —NR′R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
   each R⁵ and R⁶ is independently alkyl, alkenyl, alkynyl, cycloalkyl, aryl, heterocyclyl, or heteroaryl, each of which is independently substituted or unsubstituted, or hydrogen or halogen;
provided that, when R⁴ is —NR⁵R⁶ and one of R⁵ and R⁶ is pyridyl, then R² is not —C(O)CH₃,
   or a pharmaceutically-acceptable salt thereof.
wherein the administering is once daily for at least 4 weeks.

Embodiment 224. The method of embodiment 223, wherein R¹ is cycloalkyl.

Embodiment 225. The method of embodiment 223 or 224, wherein R¹ is cyclopentyl.

Embodiment 226. The method of any one of embodiments 223-225, wherein R² is CN.

Embodiment 227. The method of any one of embodiments 223-226, wherein R³ is hydrogen.

Embodiment 228. The method of any one of embodiments 223-227, wherein R⁴ is —NR⁵R⁶.

Embodiment 229. The method of any one of embodiments 223-228, wherein one of R⁵ and R⁶ is hydrogen.

Embodiment 230. The method of any one of embodiments 223-229, wherein one of R⁵ and R⁶ is phenyl.

Embodiment 231. The method of any one of embodiments 223-230, wherein one of R³ and Re is phenyl substituted with heterocyclyl.

Embodiment 232. The method of any one of embodiments 223-231, wherein one of R⁵ and R⁶ is phenyl substituted with piperazinyl.

Embodiment 233. The method of any one of embodiments 223-232, wherein one of R⁵ and Re is phenyl substituted with 4-methyl piperazinyl.

Embodiment 234. The method of embodiment 223, wherein R⁴ is

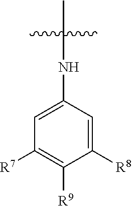

wherein:
   R⁷ is alkyl, alkenyl, alkynyl, cycloalkyl, aryl, heterocyclyl, heteroaryl, —OR⁵, —SR⁵, or —NR⁵R⁶, each of which is unsubstituted or substituted, or hydrogen;
   R⁸ is alkyl, alkenyl, alkynyl, cycloalkyl, aryl, heterocyclyl, heteroaryl, —OR⁵, —SR⁵, or —NR⁵R⁶, each of which is unsubstituted or substituted, or hydrogen; and
   R⁹ is alkyl, alkenyl, alkynyl, cycloalkyl, aryl, heterocyclyl, heteroaryl, —OR⁵, —SR⁵, or —NR⁵R⁶, each of which is unsubstituted or substituted, or hydrogen.

Embodiment 235. The method of embodiment 234, wherein R⁷ is hydrogen.

Embodiment 236. The method of embodiment 234 or 235, wherein R⁸ is hydrogen.

Embodiment 237. The method of any one of embodiments 234-236, wherein R⁹ unsubstituted or substituted heterocyclyl.

Embodiment 238. The method of any one of embodiments 234-237, wherein R⁹ is unsubstituted or substituted piperazinyl.

Embodiment 239. The method of any one of embodiments 234-238, wherein R⁹ is 4-methyl piperazinyl.

Embodiment 240. The method of any one of embodiments 234-239, wherein the compound is a compound of formula (II)

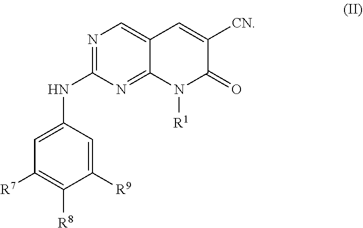

Embodiment 241. The method of any one of embodiments 234-240, wherein the compound is a compound of formula (III)

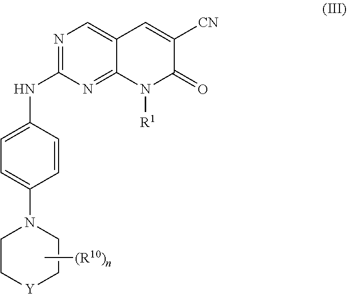

wherein:
Y is O, S, or NR¹¹;
each R¹⁰ is independently alkyl, alkenyl, alkynyl, cycloalkyl, aryl, heterocyclyl, heteroaryl, —OR⁵, —SR⁵, or —NR⁵R⁶, each of which is unsubstituted or substituted;
R¹¹ is alkyl, alkenyl, alkynyl, cycloalkyl, aryl, heterocyclyl, or heteroaryl, each of which is independently substituted or unsubstituted, or hydrogen or halogen; and
n is 0, 1, 2, 3, 4, 5, 6, 7, or 8.

Embodiment 242. The method of embodiment 241, wherein R¹ is cycloalkyl.

Embodiment 243. The method of embodiment 241 or 242, wherein R¹ is cyclopentyl.

Embodiment 244. The method of any one of embodiments 241-243, wherein Y is NR¹¹.

Embodiment 245. The method of embodiment 244, wherein R¹¹ is alkyl.

Embodiment 246. The method of embodiment 244, wherein R¹¹ is methyl.

Embodiment 247. The method of any one of embodiments 241-244, wherein n is 0.

Embodiment 248. The method of any one of embodiments 223-247, wherein the compound is 8-cyclopentyl-2-((4-(4-methylpiperazin-1-yl)phenyl)amino)-7-oxo-7,8-dihydropyrido[2,3-d]pyrimidine-6-carbonitrile, or a pharmaceutically-acceptable salt thereof.

Embodiment 249. The method of any one of embodiments 223-248, wherein the compound is 8-cyclopentyl-2-((4-(4-methylpiperazin-1-yl)phenyl)amino)-7-oxo-7,8-dihydropyrido[2,3-d]pyrimidine-6-carbonitrile monolactate.

Embodiment 250. The method of any one of embodiments 223-249, wherein the therapeutically-effective amount is about 40 mg to about 500 mg per day.

Embodiment 251. The method of any one of embodiments 223-250, wherein the ovarian cancer is a high grade serous ovarian carcinoma.

Embodiment 252. The method of any one of embodiments 223-250, wherein the ovarian cancer is a low grade serous ovarian carcinoma.

Embodiment 253. The method of embodiment 252, wherein the low grade serous ovarian carcinoma is an ovarian carcinoma.

Embodiment 254. The method of embodiment 252, wherein the low grade serous ovarian carcinoma is a fallopian carcinoma.

Embodiment 255. The method of embodiment 252, wherein the low grade serous ovarian carcinoma is a primary peritoneal carcinoma.

Embodiment 256. The method of any one of embodiments 223-255, wherein the ovarian cancer is a hormone receptor positive ovarian cancer.

Embodiment 257. The method of any one of embodiments 223-256, wherein the ovarian cancer is an estrogen receptor positive ovarian cancer.

Embodiment 258. The method of any one of embodiments 223-256, wherein the ovarian cancer is a progesterone receptor positive ovarian cancer.

Embodiment 259. The method of any one of embodiments 223-258, wherein the subject received a therapy other than the compound for the ovarian cancer prior to the administering.

Embodiment 260. The method of embodiment 259, wherein the therapy was received after the subject was diagnosed with ovarian cancer.

Embodiment 261. The method of embodiment 259, wherein the subject has not responded to the therapy.

Embodiment 262. The method of embodiment 259, wherein the subject experienced a relapse of the ovarian cancer after the therapy.

Embodiment 263. The method of any one of embodiments 223-262, wherein the administering is oral.

Embodiment 264. The method of any one of embodiments 223-262, wherein the administering is intravenous.

Embodiment 265. The method of any one of embodiments 223-264, wherein the compound is administered in a pharmaceutical composition, wherein the pharmaceutical composition is in a unit dosage form, the unit dosage form further comprising a pharmaceutically-acceptable excipient.

Embodiment 266. The method of embodiment 265, wherein the unit dosage form comprises about 40 mg of 8-cyclopentyl-2-((4-(4-methylpiperazin-1-yl)phenyl)amino)-7-oxo-7,8-dihydropyrido[2,3-d]pyrimidine-6-carbonitrile.

Embodiment 267. The method of embodiment 265 or 266, wherein unit dosage form comprises about 48.4 mg of 8-cyclopentyl-2-((4-(4-methylpiperazin-1-yl)phenyl)amino)-7-oxo-7,8-dihydropyrido[2,3-d]pyrimidine-6-carbonitrile monolactate.

Embodiment 268. The method of any one of embodiments 265-267, wherein the unit dosage form is a capsule.

Embodiment 269. The method of any one of embodiments 223-268, wherein the administering occurs in a morning of a day.

Embodiment 270. The method of any one of embodiments 223-269, wherein the subject is in a fasted state.

Embodiment 271. The method of any one of embodiments 223-268, further comprising administering to the subject a therapeutically-effective amount of a second compound.

Embodiment 272. The method of embodiment 271, wherein the second compound is an estrogen receptor modulator.

Embodiment 273. The method of embodiment 271 or 272, wherein the second compound is an estrogen receptor blocker.

Embodiment 274. The method of any one of embodiments 271-273, wherein the second compound is an aromatase inhibitor.

Embodiment 275. The method of any one of embodiments 271-274, wherein the second compound is letrozole or a pharmaceutically-acceptable salt thereof.

Embodiment 276. The method of any one of embodiments 271-275, wherein the second compound is administered in a pharmaceutical composition, wherein the pharmaceutical composition is in a unit dosage form, the unit dosage form further comprising a pharmaceutically-acceptable excipient.

Embodiment 277. The method of embodiment 276, wherein the unit dosage form comprises about 2.5 mg of letrozole.

Embodiment 278. The method of embodiment 276 or 277, the unit dosage form is a tablet.

Embodiment 279. The method of embodiment 271, wherein the second compound is a progestin.

Embodiment 280. The method of embodiment 279, wherein the progestin is megestrol acetate.

Embodiment 281. The method of embodiment 279 or 280, wherein megestrol acetate is administered in a pharmaceutical composition, wherein the pharmaceutical composition is in a unit dosage form, the unit dosage form further comprising a pharmaceutically-acceptable excipient.

Embodiment 282. The method of embodiment 281, wherein the unit dosage form comprises about 125 mg/mL of megestrol acetate.

Embodiment 283. The method of embodiment 281 or 282, wherein the unit dosage form is an oral suspension.

Embodiment 284. The method of any one of embodiments 281-283, wherein the megestrol acetate is administered at a dose of about 625 mg per day.

Embodiment 285. The method of any one of embodiments 271-284, wherein the administering of the second compound is once daily.

Embodiment 286. The method of embodiment 271, wherein the second compound is a selective estrogen receptor degrader.

Embodiment 287. The method of embodiment 271, wherein the second compound is a vascular endothelial growth factor inhibitor.

Embodiment 288. The method of embodiment 271, wherein the second compound is a phosphoinositide 3-kinase inhibitor.

Embodiment 289. The method of any one of embodiments 271-288, wherein the administering of the second compound is oral.

Embodiment 290. The method of any one of embodiments 271-288, wherein the administering of the second compound is intravenous.

Embodiment 291. The method of any one of embodiments 271-288, wherein the administering of the compound of formula (I) is oral, and the administering of the second compound is oral.

Embodiment 292. The method of any one of embodiments 271-291, herein the administering of the compound of formula (I) is prior to the administering of the second compound.

Embodiment 293. The method of any one of embodiments 271-291, wherein the administering of the second compound is prior to the administering of the compound of formula (I).

Embodiment 294. The method of any one of embodiments 271-291, wherein the administering of the compound of formula (I) is concurrently with the administering of the second compound.

Embodiment 295. A method of treating ovarian cancer in a subject in need thereof, the method comprising administering to the subject a therapeutically-effective amount of a compound of formula (I)

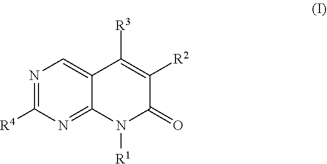

wherein:
   R¹ is alkyl, alkenyl, alkynyl, cycloalkyl, aryl, heteroaryl or heterocyclyl, each of which is unsubstituted or substituted, or hydrogen;
   R² is alkyl, alkenyl, alkynyl, cycloalkyl, aryl, heteroaryl, heterocyclyl, —CN, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen;
   R³ is alkyl, alkenyl, alkynyl, cycloalkyl, aryl, heterocyclyl or heteroaryl, each of which is unsubstituted or substituted, or hydrogen or halogen;
   R⁴ is alkyl, alkenyl, alkynyl, cycloalkyl, aryl, heterocyclyl, heteroaryl, —CN, C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
   each R⁵ and R⁶ is independently alkyl, alkenyl, alkynyl, cycloalkyl, aryl, heterocyclyl, or heteroaryl, each of which is independently substituted or unsubstituted, or hydrogen or halogen;
provided that, when R⁴ is —NR⁵R⁶ and one of R⁵ and R⁶ is pyridyl, then R² is not —C(O)CH₃,
   or a pharmaceutically-acceptable salt thereof
wherein the administering is a 4-week cycle of:
(i) a continuous, three-week period of once-daily administration; and
ii) immediately following the three-week period, one week of no administration.

Embodiment 296. The method of embodiment 295, wherein R¹ is cycloalkyl.

Embodiment 297. The method of embodiment 295 or 296, wherein R¹ is cyclopentyl.

Embodiment 298. The method of any one of embodiments 295-297, wherein R² is CN.

Embodiment 299. The method of any one of embodiments 295-298, wherein R³ is hydrogen.

Embodiment 300. The method of any one of embodiments 295-299, wherein R⁴ is —NR⁵R⁶.

Embodiment 301. The method of any one of embodiments 295-300, wherein one of R⁵ and R⁶ is hydrogen.

Embodiment 302. The method of any one of embodiments 295-301, wherein one of R⁵ and R⁶ is phenyl.

Embodiment 303. The method of any one of embodiments 295-302, wherein one of R⁵ and Re is phenyl substituted with heterocyclyl.

Embodiment 304. The method of any one of embodiments 295-303, wherein one of R⁵ and Re is phenyl substituted with piperazinyl.

Embodiment 305. The method of any one of embodiments 295-304, wherein one of R³ and R⁶ is phenyl substituted with 4-methyl piperazinyl.

Embodiment 306. The method of embodiment 295, wherein R⁴ is

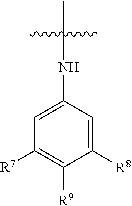

wherein:
   R⁷ is alkyl, alkenyl, alkynyl, cycloalkyl, aryl, heterocyclyl, heteroaryl, —OR⁵, —SR⁵, or —NR⁵R⁶, each of which is unsubstituted or substituted, or hydrogen;
   R⁸ is alkyl, alkenyl, alkynyl, cycloalkyl, aryl, heterocyclyl, heteroaryl, —OR⁵, —SR⁵, or —NR⁵R⁶, each of which is unsubstituted or substituted, or hydrogen; and
   R⁹ is alkyl, alkenyl, alkynyl, cycloalkyl, aryl, heterocyclyl, heteroaryl, —OR⁵, —SR⁵, or —NR⁵R⁶, each of which is unsubstituted or substituted, or hydrogen.

Embodiment 307. The method of embodiment 306, wherein R⁷ is hydrogen.

Embodiment 308. The method of embodiment 306 or 307, wherein R⁸ is hydrogen.

Embodiment 309. The method of any one of embodiments 306-308, wherein R⁹ unsubstituted or substituted heterocyclyl.

Embodiment 310. The method of any one of embodiments 306-309, wherein R⁹ is unsubstituted or substituted piperazinyl.

Embodiment 311. The method of any one of embodiments 306-310, wherein R⁹ is 4-methyl piperazinyl.

Embodiment 312. The method of any one of embodiments 306-311, wherein the compound is a compound of formula (II)

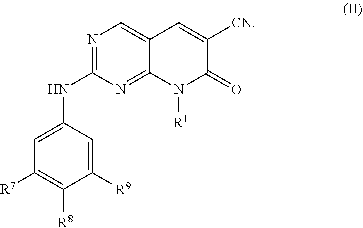

Embodiment 313. The method of any one of embodiments 306-312, wherein the compound is a compound of formula (III)

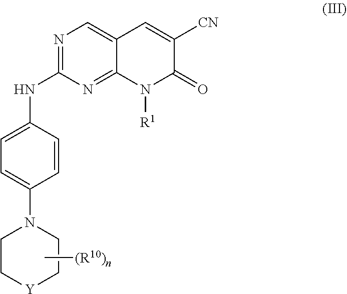

wherein:
Y is O, S, or NR¹¹;
each R¹⁰ is independently alkyl, alkenyl, alkynyl, cycloalkyl, aryl, heterocyclyl, heteroaryl, —OR⁵, —SR⁵, or —NR⁵R⁶, each of which is unsubstituted or substituted;
R¹¹ is alkyl, alkenyl, alkynyl, cycloalkyl, aryl, heterocyclyl, or heteroaryl, each of which is independently substituted or unsubstituted, or hydrogen or halogen; and
n is 0, 1, 2, 3, 4, 5, 6, 7, or 8.

Embodiment 314. The method of embodiment 313, wherein R¹ is cycloalkyl.

Embodiment 315. The method of embodiment 313 or 312, wherein R¹ is cyclopentyl.

Embodiment 316. The method of any one of embodiments 313-315, wherein Y is NR¹¹.

Embodiment 317. The method of any one of embodiments 313-316, wherein R¹¹ is alkyl.

Embodiment 318. The method of any one of embodiments 313-316, wherein R¹¹ is methyl.

Embodiment 319. The method of any one of embodiments 313-318, wherein n is 0.

Embodiment 320. The method of any one of embodiments 295-319, wherein the compound is 8-cyclopentyl-2-((4-(4-methylpiperazin-1-yl)phenyl)amino)-7-oxo-7,8-dihydropyrido[2,3-d]pyrimidine-6-carbonitrile, or a pharmaceutically-acceptable salt thereof.

Embodiment 321. The method of any one of embodiments 295-320, wherein the compound is 8-cyclopentyl-2-((4-(4-methylpiperazin-1-yl)phenyl)amino)-7-oxo-7,8-dihydropyrido[2,3-d]pyrimidine-6-carbonitrile monolactate.

Embodiment 322. The method of any one of embodiments 295-321, wherein the therapeutically-effective amount is about 40 mg to about 500 mg per day.

Embodiment 323. The method of any one of embodiments 295-322, wherein the ovarian cancer is a high grade serous ovarian carcinoma.

Embodiment 324. The method of any one of embodiments 295-322, wherein the ovarian cancer is a low grade serous ovarian carcinoma.

Embodiment 325. The method of embodiment 324, wherein the low grade serous ovarian carcinoma is an ovarian carcinoma.

Embodiment 326. The method of embodiment 324, wherein the low grade serous ovarian carcinoma is a fallopian tube carcinoma.

Embodiment 327. The method of embodiment 324, wherein the low grade serous ovarian carcinoma is a primary peritoneal carcinoma.

Embodiment 328. The method of any one of embodiments 295-327, wherein the ovarian cancer is a hormone receptor positive ovarian cancer.

Embodiment 329. The method of any one of embodiments 295-328, wherein the ovarian cancer is an estrogen receptor positive ovarian cancer.

Embodiment 330. The method of any one of embodiments 295-328, wherein the ovarian cancer is a progesterone receptor positive ovarian cancer.

Embodiment 331. The method of any one of embodiments 295-330, wherein the subject received a therapy other than the compound for the ovarian cancer prior to the administering.

Embodiment 332. The method of embodiment 331, wherein the therapy was received after the subject was diagnosed with ovarian cancer.

Embodiment 333. The method of embodiment 331, wherein the subject has not responded to the therapy.

Embodiment 334. The method of embodiment 331, wherein the subject experienced a relapse of the ovarian cancer after the therapy.

Embodiment 335. The method of any one of embodiments 295-334, wherein the administering is oral.

Embodiment 336. The method of any one of embodiments 295-334, wherein the administering is intravenous.

Embodiment 337. The method of any one of embodiments 295-336, wherein the compound is administered in a pharmaceutical composition, wherein the pharmaceutical composition is in a unit dosage form, the unit dosage form further comprising a pharmaceutically-acceptable excipient.

Embodiment 338. The method of embodiment 337, wherein the unit dosage form comprises about 40 mg of 8-cyclopentyl-2-((4-(4-methylpiperazin-1-yl)phenyl)amino)-7-oxo-7,8-dihydropyrido[2,3-d]pyrimidine-6-carbonitrile.

Embodiment 339. The method of embodiment 337, wherein unit dosage form comprises about 48.4 mg of 8-cyclopentyl-2-((4-(4-methylpiperazin-1-yl)phenyl)amino)-7-oxo-7,8-dihydropyrido[2,3-d]pyrimidine-6-carbonitrile monolactate.

Embodiment 340. The method of embodiment 337, wherein the unit dosage form is a capsule.

Embodiment 341. The method of any one of embodiments 295-340, wherein the administering occurs in a morning of a day.

Embodiment 342. The method of any one of embodiments 295-341, wherein the subject is in a fasted state.

Embodiment 343. The method of any one of embodiments 295-342, further comprising administering to the subject a therapeutically-effective amount of a second compound.

Embodiment 344. The method of embodiment 343, wherein the second compound is an estrogen receptor modulator.

Embodiment 345. The method of embodiment 343 or 344, wherein the second compound is an estrogen receptor blocker.

Embodiment 346. The method of any one of embodiments 343-345, wherein the second compound is an aromatase inhibitor.

Embodiment 347. The method of any one of embodiments 343-346, wherein the second compound is letrozole or a pharmaceutically-acceptable salt thereof.

Embodiment 348. The method of any one of embodiments 343-347, wherein the second compound is administered in a pharmaceutical composition, wherein the pharmaceutical composition is in a unit dosage form, the unit dosage form further comprising a pharmaceutically-acceptable excipient.

Embodiment 349. The method of embodiment 348, wherein the unit dosage form comprises about 2.5 mg of letrozole.

Embodiment 350. The method of embodiment 348 or 349, the unit dosage form is a tablet.

Embodiment 351. The method of embodiment 343, wherein the second compound is a progestin.

Embodiment 352. The method of embodiment 351, wherein the progestin is megestrol acetate.

Embodiment 353. The method of embodiment 352, wherein megestrol acetate is administered in a pharmaceutical composition, wherein the pharmaceutical composition is in a unit dosage form, the unit dosage form further comprising a pharmaceutically-acceptable excipient.

Embodiment 354. The method of embodiment 353, wherein the unit dosage form comprises about 125 mg/mL of megestrol acetate.

Embodiment 355. The method of embodiment 353 or 354, wherein the unit dosage form is an oral suspension.

Embodiment 356. The method of any one of embodiment 353-355, wherein the megestrol acetate is administered at a dose of about 625 mg per day.

Embodiment 357. The method of any one of embodiments embodiment 343-356, wherein the administering of the second compound is once daily.

Embodiment 358. The method of embodiment 343, wherein the second compound is a selective estrogen receptor degrader.

Embodiment 359. The method of embodiment 343, wherein the second compound is a vascular endothelial growth factor inhibitor.

Embodiment 360. The method of embodiment 343, wherein the second compound is a phosphoinositide 3-kinase inhibitor.

Embodiment 361. The method of any one of embodiments 343-360, wherein the administering of the second compound is oral.

Embodiment 362. The method of any one of embodiments 343-360, wherein the administering of the second compound is intravenous.

Embodiment 363. The method of any one of embodiments 343-360, wherein the administering of the compound of formula (I) is oral, and the administering of the second compound is oral.

Embodiment 364. The method of any one of embodiments 343-363, wherein the administering of the compound of formula (I) is prior to the administering of the second compound.

Embodiment 365. The method of any one of embodiments 343-363 wherein the administering of the second compound is prior to the administering of the compound of formula (I).

Embodiment 366. The method of any one of embodiments 343-363, wherein the administering of the compound of formula (I) is concurrently with the administering of the second compound.

Embodiment 367. A method of treating ovarian cancer in a subject in need thereof, the method comprising:
(i) administering to the subject a therapeutically-effective amount of a first compound, wherein the first compound is a compound of formula (I):

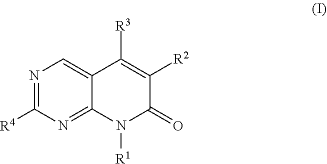

wherein:
   R¹ is alkyl, alkenyl, alkynyl, cycloalkyl, aryl, heteroaryl or heterocyclyl, each of which is unsubstituted or substituted, or hydrogen;
   R² is alkyl, alkenyl, alkynyl, cycloalkyl, aryl, heteroaryl, heterocyclyl, —CN, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen;
   R³ is alkyl, alkenyl, alkynyl, cycloalkyl, aryl, heterocyclyl or heteroaryl, each of which is unsubstituted or substituted, or hydrogen or halogen;
   R⁴ is alkyl, alkenyl, alkynyl, cycloalkyl, aryl, heterocyclyl, heteroaryl, —CN, C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
   each R⁵ and R⁶ is independently alkyl, alkenyl, alkynyl, cycloalkyl, aryl, heterocyclyl, or heteroaryl, each of which is independently substituted or unsubstituted, or hydrogen or halogen;
provided that, when R⁴ is —NR⁵R⁶ and one of R⁵ and R⁶ is pyridyl, then R² is not —C(O)CH₃,
   or a pharmaceutically-acceptable salt thereof; and
(ii) administering to the subject a therapeutically-effective amount of a second compound.

Embodiment 368. The method of embodiment 367, wherein R¹ is cycloalkyl.

Embodiment 369. The method of embodiment 367 or 366, wherein R¹ is cyclopentyl.

Embodiment 370. The method of any one of embodiments 367-369, wherein R² is CN.

Embodiment 371. The method of any one of embodiments 367-369, wherein R³ is hydrogen.

Embodiment 372. The method of any one of embodiments 367-371, wherein R⁴ is —NR⁵R⁶.

Embodiment 373. The method of any one of embodiments 367-372, wherein one of R⁵ and R⁶ is phenyl or one of R⁵ and R⁶ is hydrogen.

Embodiment 374. The method of any one of embodiments 367-372, wherein one of R⁵ and R⁶ is phenyl substituted with heterocyclyl.

Embodiment 375. The method of any one of embodiments 367-372, wherein one of R⁵ and R⁶ is phenyl substituted with piperazinyl.

Embodiment 376. The method of any one of embodiments 367-372, wherein one of R⁵ and R⁶ is phenyl substituted with 4-methyl piperazinyl.

Embodiment 377. The method of embodiment 367, wherein R⁴ is

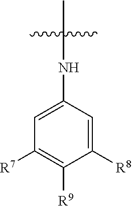

wherein:
   R⁷ is alkyl, alkenyl, alkynyl, cycloalkyl, aryl, heterocyclyl, heteroaryl, —OR⁵, —SR⁵, or —NR⁵R⁶, each of which is unsubstituted or substituted, or hydrogen;
   R⁸ is alkyl, alkenyl, alkynyl, cycloalkyl, aryl, heterocyclyl, heteroaryl, —OR⁵, —SR⁵, or —NR⁵R⁶, each of which is unsubstituted or substituted, or hydrogen; and
   R⁹ is alkyl, alkenyl, alkynyl, cycloalkyl, aryl, heterocyclyl, heteroaryl, —OR⁵, —SR⁵, or —NR⁵R⁶, each of which is unsubstituted or substituted, or hydrogen.

Embodiment 378. The method of embodiment 377, wherein R⁷ is hydrogen.

Embodiment 379. The method of embodiment 377 or 378, wherein R⁸ is hydrogen.

Embodiment 380. The method of any one of embodiments 378-379, wherein R⁹ unsubstituted or substituted heterocyclyl.

Embodiment 382. The method of any one of embodiments 378-381, wherein R⁹ is unsubstituted or substituted piperazinyl.

Embodiment 383. The method of any one of embodiments 378-382, wherein R⁹ is 4-methyl piperazinyl.

Embodiment 384. The method of any one of embodiments 378-383, wherein the compound is a compound of formula (II)

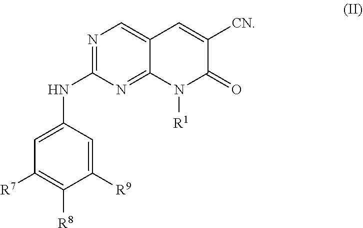

Embodiment 385. The method of any one of embodiments 378-384, wherein the compound is a compound of formula (III)

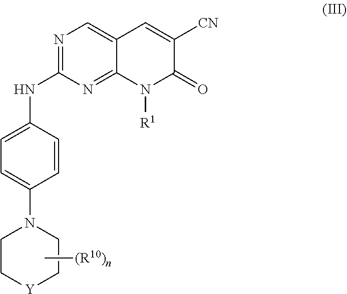

wherein:
Y is O, S, or NR¹¹;
each R¹⁰ is independently alkyl, alkenyl, alkynyl, cycloalkyl, aryl, heterocyclyl, heteroaryl, —OR⁵, —SR⁵, or —NR⁵R⁶, each of which is unsubstituted or substituted;
R¹¹ is alkyl, alkenyl, alkynyl, cycloalkyl, aryl, heterocyclyl, or heteroaryl, each of which is independently substituted or unsubstituted, or hydrogen or halogen; and
n is 0, 1, 2, 3, 4, 5, 6, 7, or 8.

Embodiment 386. The method of embodiment 385, wherein R¹ is cycloalkyl.

Embodiment 387. The method of embodiment 385 or 386, wherein R¹ is cyclopentyl.

Embodiment 388. The method of any one of embodiments 385-387, wherein Y is NR¹¹.

Embodiment 389. The method of any one of embodiments 385-388, wherein R¹¹ is alkyl.

Embodiment 390. The method of any one of embodiments 385-389, wherein R¹¹ is methyl.

Embodiment 391. The method of any one of embodiments 385-390, wherein n is 0.

Embodiment 392. The method of any one of embodiments 367-391, wherein the first compound is 8-cyclopentyl-2-((4-(4-methylpiperazin-1-yl)phenyl)amino)-7-oxo-7,8-dihydropyrido[2,3-d]pyrimidine-6-carbonitrile, or a pharmaceutically-acceptable salt thereof.

Embodiment 393. The method of any one of embodiments 367-392, wherein the first compound is 8-cyclopentyl-2-((4-(4-methylpiperazin-1-yl)phenyl)amino)-7-oxo-7,8-dihydropyrido[2,3-d]pyrimidine-6-carbonitrile monolactate.

Embodiment 394. The method of any one of embodiments 367-393, wherein the therapeutically-effective amount is about 40 mg to about 500 mg per day.

Embodiment 395. The method of any one of embodiments 367-394, wherein the ovarian cancer is a high grade serous ovarian carcinoma.

Embodiment 396. The method of any one of embodiments 367-394, wherein the ovarian cancer is a low grade serous ovarian carcinoma.

Embodiment 397. The method of embodiment 396, wherein the low grade serous ovarian carcinoma is an ovarian carcinoma.

Embodiment 398. The method of embodiment 396, wherein the low grade serous ovarian carcinoma is a fallopian tube carcinoma.

Embodiment 399. The method of embodiment 396 wherein the low grade serous ovarian carcinoma is a primary peritoneal carcinoma.

Embodiment 400. The method of any one of embodiments 367-399, wherein the ovarian cancer is a hormone receptor positive ovarian cancer.

Embodiment 401. The method of any one of embodiments 367-400, wherein the ovarian cancer is an estrogen receptor positive ovarian cancer.

Embodiment 402. The method of any one of embodiments 367-400, wherein the ovarian cancer is a progesterone receptor positive ovarian cancer.

Embodiment 403. The method of any one of embodiments 367-402, wherein the subject received a therapy other than the compound for the ovarian cancer prior to the administering of the first compound, and prior to the administering of the second compound.

Embodiment 404. The method of embodiment 403, wherein the therapy was received after the subject was diagnosed with ovarian cancer.

Embodiment 405. The method of embodiment 403, wherein the subject has not responded to the therapy.

Embodiment 406. The method of embodiment 403, wherein the subject experienced a relapse of the ovarian cancer after the therapy.

Embodiment 407. The method of any one of embodiments embodiment 367-406, wherein the administering of the first compound is oral.

Embodiment 408. The method of any one of embodiments embodiment 367-406, wherein the administering of the first compound is intravenous.

Embodiment 409. The method of any one of embodiments embodiment 367-408, wherein the administering of the first compound is once daily.

Embodiment 410. The method of any one of embodiments embodiment 367-409, wherein the administering of the first compound is once daily for at least 4 weeks.

Embodiment 411. The method of any one of embodiments embodiment 367-409, wherein the administering of the first compound is oral on a 4-week cycle of: (i) a continuous, three-week period of once-daily administration; and ii) immediately following the three-week period, one week of no administration.

Embodiment 412. The method of any one of embodiments embodiment 367-409, wherein the administering of the first compound is oral on a 4-week cycle of: (i) a continuous, three-week period of once-daily, morning administration, wherein the therapeutically-effective amount is from about 40 mg to about 500 mg; and ii) immediately following the three-week period, one week of no administration.

Embodiment 413. The method of any one of embodiments 367-412, wherein the first compound is administered in a pharmaceutical composition, wherein the pharmaceutical composition is in a unit dosage form, the unit dosage form further comprising a pharmaceutically-acceptable excipient.

Embodiment 414. The method of embodiment 413, wherein unit dosage form comprises about 40 mg of 8-cyclopentyl-2-((4-(4-methylpiperazin-1-yl)phenyl)amino)-7-oxo-7,8-dihydropyrido[2,3-d]pyrimidine-6-carbonitrile.

Embodiment 415. The method of embodiment 413 or 414, wherein unit dosage form comprises about 48.4 mg of 8-cyclopentyl-2-((4-(4-methylpiperazin-1-yl)phenyl)amino)-7-oxo-7,8-dihydropyrido[2,3-d]pyrimidine-6-carbonitrile monolactate.

Embodiment 416. The method of any one of embodiments 413-415, wherein the unit dosage form is a capsule.

Embodiment 417. The method of any one of embodiments 367-416, wherein the administering occurs in a morning of a day.

Embodiment 418. The method of any one of embodiments 367-417, wherein the subject is in a fasted state.

Embodiment 419. The method of any one of embodiments 367-418, wherein the second compound is an estrogen receptor modulator.

Embodiment 420. The method of any one of embodiments 367-419, wherein the second compound is an estrogen receptor blocker.

Embodiment 421. The method of any one of embodiments 367-420, wherein the second compound is an aromatase inhibitor.

Embodiment 422. The method of any one of embodiments 367-421, wherein the second compound is letrozole or a pharmaceutically-acceptable salt thereof.

Embodiment 423. The method of any one of embodiments 367-422, wherein the second compound is administered in a pharmaceutical composition, wherein the pharmaceutical composition is in a unit dosage form, the unit dosage form further comprising a pharmaceutically-acceptable excipient.

Embodiment 424. The method of embodiment 423, wherein the unit dosage form comprises about 2.5 mg of letrozole.

Embodiment 425. The method of embodiment 422 or 423, the unit dosage form is a tablet.

Embodiment 426. The method of embodiment 367, wherein the second compound is a progestin.

Embodiment 427. The method of embodiment 426, wherein the progestin is megestrol acetate.

Embodiment 428. The method of embodiment 427, wherein megestrol acetate is administered in a pharmaceutical composition, wherein the pharmaceutical composition is in a unit dosage form, the unit dosage form further comprising a pharmaceutically-acceptable excipient.

Embodiment 429. The method of embodiment 427 or 428, wherein the unit dosage form comprises about 125 mg/mL of megestrol acetate.

Embodiment 430. The method of any one of embodiments 427-428, wherein the unit dosage form is an oral suspension.

Embodiment 431. The method of embodiment 427-430, wherein the megestrol acetate is administered at a dose of about 625 mg per day.

Embodiment 432. The method of any one of embodiments 367-430, wherein the administering of the second compound is once daily.

Embodiment 433. The method of embodiment 367, wherein the second compound is a selective estrogen receptor degrader.

Embodiment 434. The method of embodiment 367, wherein the second compound is a vascular endothelial growth factor inhibitor.

Embodiment 435. The method of embodiment 367, wherein the second compound is a phosphoinositide 3-kinase inhibitor.

Embodiment 436. The method of any one of embodiments 367-435, wherein the administering of the second compound is oral.

Embodiment 437. The method of any one of embodiments 367-435, wherein the administering of the second compound is intravenous.

Embodiment 438. The method of any one of embodiments 367-430, wherein the administering of the first compound is oral, and the administering of the second compound is oral.

Embodiment 439. The method of any one of embodiments 367-438, wherein the administering of the first compound is prior to the administering of the second compound.

Embodiment 440. The method of any one of embodiments 367-438, wherein the administering of the second compound is prior to the administering of the first compound.

Embodiment 441. The method of any one of embodiments 367-438, wherein the administering of the first compound is concurrently with the administering of the second compound.

Embodiment 442. A method of treating ovarian cancer in a subject in need thereof, the method comprising orally administering to the subject a solid pharmaceutical composition, the solid pharmaceutical composition comprising 40 mg to 500 mg of a compound that is 8-cyclopentyl-2-((4-(4-methylpiperazin-1-yl)phenyl)amino)-7-oxo-7,8-dihydropyrido[2,3-d]pyrimidine-6-carbonitrile monolactate, wherein the subject received a therapy other than the compound for the ovarian cancer prior to the administering, wherein the therapy was received after the subject was diagnosed with ovarian cancer, and wherein the subject has not responded to the therapy prior to the administering; and wherein the administering comprises 3 weeks of once-daily administration.

Embodiment 443. A method of treating ovarian cancer in a subject in need thereof, the method comprising:
(i) orally administering to the subject a solid pharmaceutical composition, wherein the solid pharmaceutical composition comprises 40 mg to 500 mg of a compound that is 8-cyclopentyl-2-((4-(4-methylpiperazin-1-yl)phenyl)amino)-7-oxo-7,8-dihydropyrido[2,3-d]pyrimidine-6-carbonitrile monolactate,
wherein the subject received a therapy other than the compound for the ovarian cancer prior to the administering, wherein the therapy was received after the subject was diagnosed with ovarian cancer, and wherein the subject has not responded to the therapy prior to the administering; and
wherein the administering comprises 3 weeks of once-daily administration; and
(ii) orally administering to the subject a therapeutically-effective amount of letrozole.

Embodiment 444. A method of treating ovarian cancer in a subject in need thereof, the method comprising:
(i) orally administering to the subject a solid pharmaceutical composition, wherein the solid pharmaceutical composition comprises 40 mg to 500 mg of a compound that is 8-cyclopentyl-2-((4-(4-methylpiperazin-1-yl)phenyl)amino)-7-oxo-7,8-dihydropyrido[2,3-d]pyrimidine-6-carbonitrile monolactate,
wherein the subject received a therapy other than the compound for the ovarian cancer prior to the administering, wherein the therapy was received after the subject was diagnosed with ovarian cancer, and wherein the subject has not responded to the therapy prior to the administering, and
wherein the administering comprises 3 weeks of once-daily administration; and
(ii) orally administering to the subject a therapeutically-effective amount of megestrol acetate.

1.-444. (canceled)

445. A method of treating low grade serous ovarian cancer in a subject in need thereof, the method comprising administering to the subject a therapeutically-effective amount of a compound, wherein the compound is 8-cyclopentyl-2-((4-(4-methylpiperazin-1-yl)phenyl)amino)-7-oxo-7,8-dihydropyrido[2,3-d]pyrimidine-6-carbonitrile or a pharmaceutically acceptable salt thereof.

446. The method of claim 445, wherein the compound is 8-cyclopentyl-2-((4-(4-methylpiperazin-1-yl)phenyl)amino)-7-oxo-7,8-dihydropyrido[2,3-d]pyrimidine-6-carbonitrile monolactate.

447. The method of claim 445, wherein the therapeutically-effective amount is about 40 mg to about 500 mg per day.

448. The method of claim 445, wherein the low grade serous ovarian carcinoma is an ovarian carcinoma.

449. The method of claim 445, wherein the low grade serous ovarian carcinoma is a fallopian tube carcinoma.

450. The method of claim 445, wherein the low grade serous ovarian carcinoma is a primary peritoneal carcinoma.

451. The method of claim 445, wherein the low grade serous ovarian carcinoma is a hormone receptor positive low grade serous ovarian carcinoma.

452. The method of claim 445, wherein the low grade serous ovarian carcinoma is an estrogen receptor positive low grade serous ovarian carcinoma.

453. The method of claim 445, wherein the low grade serous ovarian carcinoma is a progesterone receptor positive low grade serous ovarian carcinoma.

454. The method of claim 445, wherein the administering is oral.

455. The method of claim 445, wherein the administering is intravenous.

456. The method of claim 445, further comprising administering to the subject a therapeutically-effective amount of a second compound.

457. The method of claim 456, wherein the second compound is an estrogen receptor modulator.

458. The method of claim 456, wherein the second compound is an estrogen receptor blocker.

459. The method of claim 456, wherein the second compound is an aromatase inhibitor.

460. The method of claim 456, wherein the second compound is letrozole or a pharmaceutically-acceptable salt thereof.

461. The method of claim 456, wherein the second compound is a progestin.

462. The method of claim 461, wherein the progestin is megestrol acetate.

463. The method of claim 456, wherein the second compound is a selective estrogen receptor degrader.

464. The method of claim 456, wherein the second compound is a vascular endothelial growth factor inhibitor.

465. The method of claim 456, wherein the second compound is a phosphoinositide 3-kinase inhibitor.
